(19) United States
(12) Patent Application Publication
(10) Pub. No.: US 2025/0306034 A1
(43) Pub. Date: Oct. 2, 2025

(54) Detection of Liver Disease

(71) Applicant: Owlstone Medical Limited, Cambridge (GB)

(72) Inventors: Giuseppe FERRANDINO, Cambridge (GB); Olga GANDELMAN, Cambridge (GB); Max Allsworth, Cambridge (GB); Marc van der Schee, Cambridge (GB)

(21) Appl. No.: 18/866,414

(22) PCT Filed: May 18, 2023
(86) PCT No.: PCT/GB2023/051313

§ 371 Date: Nov. 15, 2024

(30) Foreign Application Priority Data
May 18, 2022 (GB) 2207291.2

(51) Int. Cl. G01N 33/68

(52) U.S. Cl. CPC G01N 33/6893; G01N 2800/085; G01N 2800/52; G01N 2800/56

(57) ABSTRACT

This invention relates to methods of detecting, staging, monitoring or prognosing non-alcoholic steatohepatitis (NASH) in a subject. The method comprises measuring the concentration of an exogenous substrate for an enzyme and/or measuring the concentration of a metabolite of said substrate in a biological sample obtained from said subject. The substrate is a generally recognised as safe (GRAS) compound.

### FIELD OF THE INVENTION

The invention relates to methods for the detection of non-alcoholic steatohepatitis (NASH) and related methods and kits.

### INTRODUCTION

Nonalcoholic Fatty Liver Disease (NAFLD) encompasses an entire histologic spectrum ranging from simple, benign hepatic steatosis to non-alcoholic steatohepatitis (NASH) characterized by lipid accumulation, inflammation, hepatocyte ballooning, and varying degrees of fibrosis. NASH may progress to cirrhosis or hepatocellular carcinoma (HCC). NASH, unlike non-alcoholic fatty liver (NAFL), has the greatest potential to progress to cirrhosis, liver failure, and liver cancer. The prevalence of NAFLD is increasing and is linked to the increase in cases of obesity. Despite increasing awareness of obesity-related liver disease, the pathogenesis of NAFLD and NASH remain poorly understood.

Based on the prevalence of NAFLD, it is anticipated that NASH-induced cirrhosis will become the most common indication for liver transplantation in the future. Differentiating NASH from simple steatosis is important for the clinical management of NAFLD patients and to reduce mortality (Chen et al, Radiology. 2011 June; 259 (3): 749-756).

The sole test approved for NASH diagnosis is liver biopsy, an invasive procedure that can lead to complications. Surrogate methods lack adequate performance in early NASH stages and overall, this limits NASH early detection and makes it difficult to evaluate the efficacy of experimental drugs. Thus, there is a need for alternative diagnostic tests, in particular tests that can diagnose NASH and differentiate NASH from other stages of NAFLD, i.e. NAFL. Identification of both NAFLD and NASH non-invasively would help to significantly reduce the risk associated with diagnosis of these pathologies. Differentiating between NASH and NAFL allows for earlier lifestyle changes, medical interventions, cancer screening, and overall improved outcomes.

The use of exogenous volatile organic compound (EVOC®) probes for induced volatolomics—monitoring the metabolic processing of an exogenous compound by monitoring exhaled breath—to detect liver disease has been described in WO2019220145. The use of probes as described herein for NASH detection, diagnostics, staging, monitoring and prognosis provides alternative tests for NASH.

### SUMMARY OF THE INVENTION

The inventors have shown that NASH-induced metabolic alterations are detectable using EVOC Probes, which are safe for human consumption and appear in breath after administration, together with their bioproducts. These data demonstrate that breath analysis using EVOC Probes for induced volatolomics can be used in a NASH detection test as well as for screening, diagnostics, staging, monitoring and prognosis.

In this approach, one or more compounds of interest, which appear in the exhaled breath of a subject and are metabolized by the liver, are measured on breath after administration. The amount of compound(s) in a subject's breath depends on the efficiency of the liver in clearing the compound after administration. Therefore, the amount in breath represents liver function. Volatile bioproducts can be detected either alternatively or additionally to increase diagnostic accuracy. Therefore, the amount of compound(s) in breath can be used as a proxy of liver function, which is affected in NASH.

As such the present inventors have developed a non-invasive test to detect and stage non-alcoholic fatty liver disease (NAFLD) progressed to the stage of non-alcoholic steatohepatitis (NASH), in a subject with or suspected of having liver disease for detection, diagnostics, staging, monitoring and prognosis (i.e., at risk of progression to a more advanced liver disease stage).

In a first aspect, the invention thus relates to a method for detecting or prognosing NASH in a subject, comprising measuring the concentration of an exogenous substrate for an enzyme and/or measuring the concentration of a metabolite of said substrate in a biological sample obtained from said subject wherein the substrate is a generally recognised as safe (GRAS) compound and wherein the enzyme is an aldo-ketoreductase (AKR) or an alcohol dehydrogenase or a Cytochrome P450 (CYP) or aldehyde dehydrogenase or glycine N-acyltransferase.

The method further comprises determining the stage of NASH, wherein the NASH stage is selected from NASH without fibrosis, NASH with fibrosis, NASH with hepatocellular carcinoma (HCC) or NASH with cirrhosis, such as decompensated cirrhosis.

In one embodiment, the enzyme is an aldo-ketoreductase (AKR).

In one embodiment, the AKR is an AKR family 1 member.

In one embodiment, the AKR is AKR1B10. In one embodiment, the substrate is selected from an aldehyde and/or an alcohol.

In one embodiment, the substrate is nonanal and/or the metabolite is nonanol.

In one embodiment, the substrate is 1-nonanal and/or the metabolite is 1-nonanol.

In one embodiment, the substrate is trans-2-hexenal and/or the metabolite is trans-2-hexanol.

In one embodiment, wherein the substrate is hexanal and/or the metabolite is hexanol.

In one embodiment, the substrate is benzyl aldehyde and/or the metabolite is benzyl alcohol.

In one embodiment, the substrate is citral and/or the metabolite is nerol.

In one embodiment, the enzyme is an alcohol dehydrogenase.

In one embodiment, the substrate is butanol and/or the metabolite is butanone.

In one embodiment, the substrate is 2-butanol and/or the metabolite is 2-butanone.

In one embodiment, the substrate is 2-pentanone.

In one embodiment, the substrate is 2-pentanone and/or the metabolite is 2-pentanol, 3-hydroxy-2-pentanone or 2,3-pentanediol.

In one embodiment, the substrate is benzyl alcohol and the metabolites are benzylaldehyde and/or benzoic acid.

In one embodiment, the enzyme is aldehyde dehydrogenase.

In one embodiment, the substrate is benzaldehyde and the metabolite is benzoic acid.

In one embodiment, the enzyme is a Cytochrome P450 (CYP).

In one embodiment the CYP is CYP1A1, CYP1A2, CYP1 B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7 or CYP3A43. In one embodiment, the enzyme is CYP2C19, CYP2C9 and/or CYP3A4.

In one embodiment, the substrate is 2-butanone and the metabolite is 3-hydroxy-2-butanone and/or 2,3-butanediol.

In one embodiment, the substrate is 2-pentanone and the metabolite is 2,3-pentanediol.

In one embodiment, the enzyme is glycine N-acyltransferase.

In one embodiment, the substrate is benzoic acid and the metabolite is hippuric acid.

In one embodiment, the substrate is labelled, for example with 12C, 13C, 14C, 2H, 14N or 18O.

In one embodiment, the substrate is not labelled.

In one embodiment, the biological sample is selected from breath, urine, blood, serum, and/or tissue.

In one embodiment, the method comprises establishing a test subject value based on a concentration of said substrate or metabolite in said test subject.

In one embodiment, the test subject value is compared to one or more reference values and wherein a difference in the test subject value and a reference value indicates a likelihood of NASH.

In one embodiment, the reference value is the value of a subject that has been diagnosed with NASH.

In one embodiment, the reference value is the value of a subject that has been diagnosed with non-alcoholic fatty liver disease (NAFLD).

In one embodiment, the reference value is the value of a subject with NASH that has progressed to decompensated cirrhosis and/or HCC.

In one embodiment, the reference value is the value of a healthy subject.

In one embodiment, the concentration of two or more exogenous substrates for the enzyme and/or the concentration of two or more metabolites is measured.

In one embodiment, the subject has been administered the exogenous substrate for the enzyme.

In one embodiment, the concentration of the metabolite is measured.

In another aspect, the invention relates to a method for determining efficacy of a treatment comprising in a subject diagnosed with NASH, assessing the activity of an enzyme by measuring the concentration of an exogenous substrate for said enzyme and/or measuring the concentration of a metabolite of said substrate in a biological sample obtained from the subject, wherein said subject has received treatment for NASH and wherein the enzyme is an aldo-ketoreductase (AKR) or an alcohol dehydrogenase or a Cytochrome P450 (CYP).

In one embodiment, the method comprises analysing a first biological sample obtained from said subject at a first time point, and then analysing one or more additional biological samples obtained from said subject at one or more additional time points or ratios thereof.

In one embodiment, said treatment of NASH is gastric bypass surgery, and/or a drug-based treatment comprising the administration of at least one drug.

In one embodiment, said treatment of NASH is gastric bypass surgery, and/or a drug-based treatment comprising the administration of at least one drug selected from statins, incretin analogues, metformin, rimonabant, thiazolidinediones, and orlistat.

In another aspect, the invention relates to a method of monitoring the progression or regression of NASH in a subject, comprising measuring the concentration of an exogenous substrate for an enzyme and/or measuring the concentration of a metabolite of said substrate in a biological sample obtained from a subject wherein the substrate is a generally recognised as safe (GRAS) compound and wherein the enzyme is an aldo-ketoreductase (AKR) or an alcohol dehydrogenase or a Cytochrome P450 (CYP).

In another aspect, the invention relates to a kit for the detection or prognosis of NASH comprising substrate for an enzyme and/or the metabolite of said substrate and a device for capturing a biological sample from a patient.

In one embodiment, said substrate and/or metabolite is selected from nonanal, butanol, trans-2-hexenal, hexanal, benzaldehyde, citral, nonanol, butanone, trans-2-hexenol, hexanol, benzyl alcohol, nerol, 3-hydroxy-2-butanone, 2,3-butanediol, benzoic acid, hippuric acid, 2-pentanone and 2,3-pentanediol.

In another aspect, the invention relates to a use of an exogenous substrate and or metabolite for an enzyme whose activity or expression is upregulated or downregulated in NASH in a method for detecting or prognosing NASH, wherein said substrate is selected from nonanal, butanol, trans-2-hexenal, hexanal, citral, benzoic acid, butanone, and 2-pentanone, and said metabolite is selected from nonanol, butanone, trans-2-hexenol, hexanol, benzyl alcohol, nerol, 2-pentanone, 3-hydroxy-2-butanone, 2,3-butanediol, hippuric acid, and 2,3-pentanediol.

In another aspect, the invention relates to nonanal, butanol, trans-2-hexenal, hexanal, benzaldehyde, citral, 2-pentanone, nonanol, butanone, trans-2-hexenol, hexanol, benzyl alcohol, nerol, 2-pentanol, benzoic acid, hippuric acid, 2,3-butanediol, 3-hydroxy-2-pentanone and/or 2,3-pentanediol for use in an in vivo method of detecting or prognosing NASH in a subject, comprising measuring the concentration of nonanal, butanol, trans-2-hexenal, hexanal, benzaldehyde, citral, 2-pentanone, nonanol, butanone, trans-2-hexenol, hexanol, benzyl alcohol, nerol, 2-pentanol, benzoic acid, hippuric acid, 2,3-butanediol, 3-hydroxy-2-pentanone 5 and/or 2,3-pentanediol in a biological sample obtained from said subject.

In another aspect, the invention relates to a use of nonanal, butanol, trans-2-hexenal, hexanal, benzaldehyde, citral, 2-pentanone, nonanol, butanone, trans-2-hexenol, hexanol, benzyl alcohol, nerol, 2-pentanol, benzoic acid, hippuric acid, 2,3-butanediol, 3-hydroxy-2-pentanone and/or 2,3-pentanediol as a biomarker for NASH detection, staging (i.e. assessing the stage of NASH), diagnosis, monitoring and progression.

In another aspect, the invention relates to a method of differentiating between NASH and other stages of NAFLD in a subject, comprising measuring the concentration of an exogenous substrate for an enzyme and/or measuring the concentration of a metabolite of said substrate in a biological sample obtained from a subject wherein the substrate is a generally recognised as safe (GRAS) compound.

In one embodiment, the activity or expression of said enzyme is upregulated or downregulated in NASH.

In one embodiment the enzyme is an aldo-ketoreductase (AKR) or an alcohol dehydrogenase or a Cytochrome P450 (CYP).

In another aspect, the invention relates to a method for detecting or prognosing early-stage non-alcoholic steatohepatitis (NASH) in a subject, comprising measuring the concentration of an exogenous substrate for an enzyme and/or measuring the concentration of a metabolite of said substrate in a biological sample obtained from said subject wherein the substrate is a generally recognised as safe (GRAS) compound. In one embodiment, the activity or expression of said enzyme is upregulated or downregulated in NASH. In one embodiment the enzyme is an aldo-ketoreductase (AKR) or an alcohol dehydrogenase or a Cytochrome P450 (CYP).

In another aspect, the invention relates to a method of determining the stage of NASH in a subject, comprising measuring the concentration of an exogenous substrate for an enzyme and/or measuring the concentration of a metabolite of said substrate in a biological sample obtained from a subject wherein the substrate is a generally recognised as safe (GRAS) compound. In one embodiment, the activity or expression of said enzyme is upregulated or downregulated in NASH.

In one embodiment the enzyme is an aldo-ketoreductase (AKR) or an alcohol dehydrogenase or a Cytochrome P450 (CYP).

In another aspect of the invention, the invention relates to a method of determining the stage of NASH in a subject, comprising measuring the concentration of an exogenous substrate for an enzyme and/or measuring the concentration of a metabolite of said substrate in a biological sample obtained from a subject wherein the substrate is a generally recognised as safe (GRAS) compound.

In one embodiment, the activity or expression of said enzyme is upregulated or downregulated in NASH.

In one embodiment, the enzyme is an aldo-ketoreductase (AKR) or an alcohol dehydrogenase or a Cytochrome P450 (CYP).

In another, the invention thus relates to a method for detecting or prognosing NASH in a subject, detecting or prognosing early stage NASH in a subject, determining the stage of NASH in a subject, monitoring the progression or regression of NASH in a subject or differentiating between NASH and other stages of NAFLD in a subject comprising measuring the concentration of an exogenous substrate for an enzyme and/or measuring the concentration of a metabolite of said substrate in a biological sample obtained from said subject wherein the substrate is a generally recognised as safe (GRAS) compound and wherein the enzyme is not a CYP enzyme.

In another aspect, the invention relates to a method for detecting or prognosing NASH in a subject, detecting or prognosing early stage NASH in a subject, determining the stage of NASH in a subject, monitoring the progression or regression of NASH in a subject or differentiating between NASH and other stages of NAFLD in a subject comprising measuring the concentration of an exogenous substrate for an enzyme and/or measuring the concentration of a metabolite of said substrate in a biological sample obtained from said subject wherein the substrate is a generally recognised as safe (GRAS) compound wherein the substrate is not limonene.

In another aspect, the invention relates to a method for determining efficacy of a treatment comprising in a subject diagnosed with NASH, assessing the activity of an enzyme by measuring the concentration of an exogenous substrate for said enzyme and/or measuring the concentration of a metabolite of said substrate in a biological sample obtained from the subject, wherein the enzyme is not a CYP enzyme or wherein the substrate is not limonene.

In all of the aspects above, combinations of substrates and/or metabolites can be used as explained further below.

### BRIEF DESCRIPTION OF THE FIGURES

FIG. 1. Western blot analysis of cell extracts obtained from wild type (WT) or AKR1B10 CRISP/Cas knock out (KO).

FIG. 2. Headspace analysis of WT and KO cells. 2A and B. Medium supplemented with 30 UM nonanal; 2C and D. Medium supplemented with 10 UM trans-2-hexenal.

FIG. 3. Washouts in healthy volunteers.

FIG. 4. Detection of nonanal and nonanol from healthy human hepatocytes.

FIG. 5. Detection of butanol and butanone from healthy human hepatocytes.

FIG. 6A. Lactate dehydrogenase level (LDH) measured in the supernatant of healthy and NASH hepatocytes. 6B. Serum albumin measured in the supernatant of healthy and NASH hepatocytes. 6C. Levels of interleukin 6 (IL-6). 6D. Levels of TIMP Metallopeptidase Inhibitor 1 (TIMP-1).

FIG. 7. Microscopy image of cultured hepatocytes.

FIG. 8. Analysis of NASH and healthy hepatocytes. 8A. Levels of 2-butanol. 8B. Levels of 2-butanone. 8C. Levels of nonanal. 8D. Levels of nonanol.

FIG. 9. A) Timeline of NASH rat model showing rat ages in weeks old and action taken at each timepoint. B) Diagram showing the numbers of Wistar Han rats given a normal diet and given a choline deficient high fat diet. C) Boxplot of fibrosis area, by disease demonstrating that rats fed choline deficient high fat diet exhibit symptoms of NASH.

FIG. 10. Boxplots of rat weights, by disease and days since diet started.

FIG. 11. Boxplots of benzoic acid, by time and disease.

FIG. 12. Boxplot of 3-hydroxy-2-butanone, by time (min) and disease.

FIG. 13. Boxplot of 2,3-butanediol, by time (min) and disease

FIG. 14. Boxplot of 2,3-pentanediol, by time (min) and disease.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Methods

The present invention provides methods of detecting, staging, screening, diagnosing, monitoring or prognosing NAFLD progressed to the stage of NASH. Advantageously the present inventors have developed a method of detecting NASH which may be non-invasive or minimally invasive as the method is performed on a biological sample. Furthermore, the inventors have developed a method to identify the stage of the disease, i.e. how far the disease has progressed as well as differentiating between NASH and NAFL.

In a first aspect the invention relates to a method for detecting or prognosing NAFLD progressed to the stage of NASH, in a subject, comprising measuring the concentration of an exogenous substrate for a NASH specific enzyme and/or measuring the concentration of a metabolite of said substrate in a biological sample obtained from said subject wherein the substrate is a generally recognised as safe (GRAS) compound and wherein the enzyme is an aldo-ketoreductase (AKR) or an alcohol dehydrogenase or a Cytochrome P450 (CYP) or aldehyde dehydrogenase or glycine N-acyltransferase.

NAFLD is a term that refers to a range of conditions which are caused by a build-up of fat in the liver. The term encompasses a disease spectrum which includes a mild benign form of the disease where there is a build-up of fat in the liver, referred to as steatosis or NAFL. This disease state can progress to a more severe form known as NASH wherein the liver becomes inflamed, this stage may also be referred to herein as NASH without fibrosis. NASH can then progress to NASH with fibrosis, where persistent inflammation causes scar tissue around the liver and nearby blood vessels to form. The most severe form of the disease is NASH with cirrhosis which can occur after long term inflammation, resulting in shrinkage and scarring of the liver. NASH with cirrhosis causes permanent damage to the liver and can lead to liver failure and development of liver cancer (hepatocellular carcinoma (HCC)).

The severity of NAFLD can be described among three stages. Stage 1 is characterized by simple fatty liver (i.e. NAFL or hepatic steatosis). Fat begins to accumulate in individual cells but liver function is normal. There are usually no symptoms and patients may not realize they have the condition. Although the fat deposits are considered harmless, it is important to prevent the disease from progressing to the next stage.

Stage 2 is often referred to as NASH. NASH is a more aggressive form of the condition, where the liver has become inflamed. Inflammation is the body's healing response to damage or injury and, in this case, is a sign that liver cells have become damaged. A person with NASH may have a dull or aching pain felt in the top right of their abdomen (over the lower right side of their ribs). NASH can occur with or without fibrosis.

Stage 3 is often characterized by cirrhosis. At this most severe stage, bands of scar tissue and clumps of liver cells develop. The liver shrinks and becomes lumpy which is known as cirrhosis. Cirrhosis progresses slowly gradually causing the liver to stop functioning. The damage caused by cirrhosis is irreversible and the patient may experience signs of liver failure. Cirrhosis tends to occur after the age of 50, usually after years of liver inflammation associated with the early stages of the disease. People with cirrhosis of the liver caused by NAFLD often also have type 2 diabetes.

In an embodiment the method further comprises determining the stage of NASH, wherein the NASH stage is selected from: NASH without fibrosis (that is progressed to fibrosis) or NASH with fibrosis. In some embodiments, NASH induced with cirrhosis, e.g. decompensated cirrhosis or hepatocellular carcinoma (HCC) are also within the scope of the invention.

The term “cirrhosis,” “liver cirrhosis” or “hepatic cirrhosis” refers to a condition in which the liver does not function properly due to long-term damage. This damage is characterized by the replacement of normal liver tissue by scar tissue (i.e. fibrosis). The disease generally develops slowly over months or years, often with no symptoms. Eventually, excessive scar formation will result in loss of liver function.

The term “prognosis” refers to the forecast or likely outcome of a disease. As used herein, it refers to the probable outcome of liver disease, including whether the disease (e.g. NASH) will respond to treatment or mitigation efforts and/or the likelihood that the disease will progress.

The term progression as used herein may refer to an advancement of the disease state. The term regression as used herein may refer to a decrease of the severity of the disease state. When the disease is monitored, this can result in detecting progression or regression.

Regression may be due to health style changes or therapeutic intervention, for example using a treatment as described herein, including a treatment in clinical drug trials.

In one embodiment, the stage is early-stage NASH. Early-stage NASH is different from NAFL and is characterised by steatosis, inflammation, high hepatic fat and hepatocellular injury.

The present method is based on administration of an exogenous substrate to a subject. An “exogenous substrate” is any compound that can be administered to a subject that is metabolised by an enzyme within the subject. An exogenous substrate refers to a chemical compound that is recognized by the enzyme of interest and for which the enzyme catalyzes conversion of the substrate into a different chemical compound which is referred to herein as a “metabolite”. The substrate used in the methods of the invention is an exogenous substance, i.e. a xenobiotic. The term xenobiotic refers to a substance that is foreign to the subject's body and which is specifically and selectively metabolised by the enzyme. Preferably, the exogenous substance converted into a metabolite by the enzyme is also a xenobiotic, that does not normally occur in the subject's body. In an embodiment the exogenous substrate is a generally recognised as safe (GRAS) compound.

The exogenous substrate is selectively metabolised by an enzyme within the subject. The enzyme is an enzyme whose activity or expression are downregulated in NASH or whose activity or expression are upregulated in NASH, that is in patients that present with NASH disease, compared to healthy subjects. For example, activity or expression are upregulated or downregulated in liver tissue in NASH patients compared to healthy subjects. Thus, the changes in expression and/or activity of the enzyme are indicative of NASH.

For example, the gene encoding for the enzyme may be differentially expressed in NASH tissue compared to non-NASH tissue. For example, the enzyme may be expressed at a higher level in NASH tissue compared to non-NASH tissue or at a lower level in NASH tissue compared to non-NASH tissue. In another embodiment, the enzyme may be differentially active in NASH tissue compared to non-NASH tissue. For example, the enzyme may be modified such that the activity of the enzyme is higher or lower in NASH tissue compared to the activity in non-NASH tissue. Gene expression can be measured by techniques known in the art, for example by mRNA quantification or measuring cDNA. The activity of an enzyme can be measured by evaluating its metabolic activity, that is the enzyme's capacity to metabolise a substrate.

Non-NASH tissue may refer for example to healthy tissue or to NAFLD tissue which has not progressed to NASH for example tissue from a subject with NAFL also referred to as steatosis. The tissue may be from a specific organ, e.g. liver, lung, colon, breast, prostate etc. In one embodiment, the tissue is liver tissue.

The methods of the invention may include an additional step of identifying a suitable enzyme whose activity or expression are downregulated in NASH or whose activity or expression are upregulated in NASH compared to non-NASH tissue. The methods of the invention may include a further step of identifying a substrate of the enzyme and optionally the metabolite produced due to the enzymatic action.

Thus, methods described herein indirectly measure the activity of enzymes that are directly associated with a NASH disease state in a non-invasive or minimally invasive way by measuring the activity of the enzymes via the metabolism of substrates in a biological sample. Due to the association between the enzyme and its ability in breaking down a substrate and the NASH disease state, a diagnosis or prognosis can be made as to the patient's disease state. On that basis, a suitable treatment can be selected. In particular, treatment may be selected from one or more of gastric bypass surgery, and/or a drug-based treatment comprising the administration of at least one drug selected from statins, incretin analogues, metformin, rimonabant, thiazolidinediones, and orlistat.

In one embodiment, the methods of the invention include a step of administering a suitable treatment to treat NASH disease following a diagnosis that the subject has NASH or is at risk of developing NASH. Thus, the invention also provides a method for treating NASH in a subject, comprising measuring the concentration of an exogenous substrate for an enzyme and/or measuring the concentration of a metabolite of said substrate in a biological sample obtained from said subject wherein the substrate is a GRAS compound and wherein the enzyme is an aldo-ketoreductase (AKR) or an alcohol dehydrogenase and comprising treating the subject.

Metabolism and transformation of the substrate by one or more enzyme leads to the generation of a breakdown product, that is a metabolic product, i.e. a metabolite. Soon after provision of the substrate to the subject, the substrate is excreted into biological matrices such as breath, urine, blood at high levels and clearance of the substrate from said biological matrices occurs as a consequence of biotransformation of the substrate by the action of one or more enzymes (washout of the reactant). For example, the kinetic profile of the clearance of the substrate from breath may be used as a readout of the enzyme activity responsible for biotransformation of said substrate.

In addition, metabolism of a specific substrate through one or more enzyme leads to production of enzyme-specific metabolic products. In this case metabolic products are excreted into biological matrices over time, starting at low levels and increasing over time due to biotransformation of the substrate by the enzyme. Measurement of such a metabolic product can be applied as a probe for assessing the metabolic phenotype of the enzyme or enzymes responsible for the production of said product.

As explained further herein and in the examples, the wash-out curves for certain metabolites are different between NASH and non-NASH tissue, depending on whether the enzyme that metabolises the substrate is overexpressed or downregulated in NASH tissue.

For example, healthy hepatocytes showed higher production of 2-butanone (from the breakdown of butanol) at 6 hours and the area under the peak for healthy is larger than for NASH, as expected given that the alcohol dehydrogenase pathway involved in the conversion of 2-butanol to 2-butanone is downregulated in NASH (see examples).

For nonanol production from the breakdown of nonanal, NASH hepatocytes showed higher production of nonanol compared to healthy hepatocytes (the area of the peak is larger in NASH than in healthy). This result aligns with the overexpression of AKR1B10, the enzyme that converts nonanal to nonanol, observed in NASH liver (see examples).

In an embodiment the enzyme used in the methods of the invention is an aldo-ketoreductase (AKR). AKRs are enzymes that catalyze redox transformations involved in biosynthesis, intermediary metabolism and detoxification.

AKRs catalyse oxidation-reduction reactions on a wide variety of substrates including glucocorticoids, carbonyl metabolites, glutathione conjugates, and phospholipid aldehydes, among others (Barski, Tipparaju, and Bhatnagar 2008. Aldo-Keto Reductase Superfamily and Its Role in Drug Metabolism and Detoxification. Drug Metabolism Reviews 40 (4)). Given the wide diversity of biological substrates, it appears that AKRs may have the common function of detoxification of aldehydes and ketones produced by endogenous metabolic reactions, as well as environmental toxins encountered via food, medications or other sources (Bachur 1976, Cytoplasmic Aldo-Keto Reductases: A Class of Drug Metabolizing Enzymes. Science 193 (4253): 595-97). Using pyridine nucleotides as cofactors, most AKRs catalyse reduction of aldehydes and ketones, while being relatively inefficient alcohol dehydrogenases (Barski, Tipparaju, and Bhatnagar 2008).

Most of the energy required for carbonyl reduction by AKRs is obtained from nucleotide cofactor binding, rather than substrate binding, resulting in highly efficient reduction of substrates, even when loosely bound to the active site. This explains the wide range of substrates that some AKR families, such as AKR1 B family, can act on (Grimshaw 1992, Aldose Reductase: Model for a New Paradigm of Enzymic Perfection in Detoxification Catalysts. Biochemistry 31 (42): 10139-45) and justifies the pivotal role of AKRs as detoxifying enzymes. The carbonyl group present in aldehydes is very reactive and can readily attack nucleophilic centres, such as protein aminoacids and membrane phospholipids.

The reduction of aldehydic carbonyls into alcohols by AKRs reduces the overall chemical reactivity of the molecule, and is one of the mechanisms of detoxification of reactive aldehydes from the cell (Barski, Tipparaju, and Bhatnagar 2008). Lipid peroxidation can give rise to a wide range of different toxic aldehydes, because ROS can oxidise any bisallylic group in the lipid chain (Ayala, Munoz, and ArgOelles 2014, 2014. Lipid Peroxidation: Production, Metabolism, and Signaling Mechanisms of Malondialdehyde and 4-Hydroxy-2-Nonenal. Oxidative Medicine and Cellular Longevity; Yin, Xu, and Porter 2011. Free Radical Lipid Peroxidation: Mechanisms and Analysis. Chemical Reviews 111 (10): 5944-72). Furthermore, it is considered that aldehydes are the major by-product of lipid peroxidation (Barski, Tipparaju, and Bhatnagar 2008). The wide range of substrate-specificity, together with the ability to readily reduce reactive aldehydes, makes the AKRs family an ideal antioxidant mechanism against lipid peroxidation and ferroptosis. In support of this hypothesis, AKRs have been shown to reduce products of lipid peroxidation, such as 4-hydroxynonenal (Gimenez-Dejoz et al. 2015 Substrate Specificity, Inhibitor Selectivity and Structure-Function Relationships of Aldo-Keto Reductase 1: A Novel Human Retinaldehyde Reductase.” PLOS ONE 10 (7);), as well as PAPC and POVPC (Srivastava et al. 2004, Aldose Reductase-Catalyzed Reduction of Aldehyde Phospholipids. Journal of Biological Chemistry 279 (51)).

Members of the AKR1 B family have been shown to act on several, highly volatile, compounds. Indeed AKR1B1, AKR1 B10 and AKR1 B15 have shown substrate-specificity for the volatile aldehydes benzaldehyde and cinnamaldehyde, the alkanal hexanal, the alkenals 4-hydroxynonenal, hexenal, and farnesal, the ketones 3-nonen-2-one, and the dicarbonyls 2,3-butanedione and 2,3-hexanedione, among others (Gimenez-Dejoz et al. 2015).

Aldo-keto reductase family 1 member B10 (AKR1B10) is associated with HCC and is secreted into the blood by liver cells via a lysosome-mediated nonclassical pathway. Secretion of AKR1B10 protein is associated with advanced NASH (Kanno, M. et al. 2019. Serum aldo-keto reductase family 1 member B10 predicts advanced liver fibrosis and fatal complications of nonalcoholic steatohepatitis. J Gastroenterol 54, 549-557).

In an embodiment, the enzyme may be a AKR family 1 member in particular it may be AKR family 1 member B10 (AKR1B10).

In another embodiment, the enzyme used in the method of the invention is an alcohol dehydrogenase. Alcohol metabolism is a well-characterized biological process that is dominated by the alcohol dehydrogenase (ADH) and aldehyde dehydrogenase (ALDH) families.

Alcohol dehydrogenases catalyse the oxidation of primary and secondary alcohols to the corresponding aldehyde or ketone. Alterations in alcohol metabolism processes in response to human NASH progression have been investigated and the activity and expression of Alcohol dehydrogenase enzymes has been studied (Li, H., Toth, E. & Cherrington, N. J. Alcohol Metabolism in the Progression of Human Nonalcoholic Steatohepatitis. Toxicol Sci 164, 428-438, (2018)).

In an embodiment, the enzyme is aa-alcohol dehydrogenase (aaADH). In one embodiment, the enzyme is ALDH4A1, ADH1A, ADH1B, ADH4, and ALDH2.

The exogenous substrate used in the present methods is specific for the enzyme such that the substrate is selectively metabolised by the enzyme. The exogenous substrate may therefore be any substrate that is suitable for detecting the enzyme activity. In an embodiment, the substrate and/or its metabolite is a VOC that is secreted in biological matrices, preferably a VOC that is secreted into biological matrices at high proportions. Generally, VOCs are defined as organic chemical compounds whose composition makes it possible for them to evaporate under normal indoor atmospheric conditions of temperature and pressure. Since the volatility of a compound is generally higher the lower its boiling point temperature, the volatility of organic compounds is sometimes defined and classified by their boiling points. Volatile compounds are compounds that are secreted by the human body into gas fluids, including for example breath, skin emanations and others. Optionally the substrate and/or metabolite is a VOC that can be measured in a biological matrix without the use of any labels, such as isotope labels. Preferably, the exogenous substrate may be selected from an aldehyde and/or an alcohol.

According to the various aspects of the invention, in one embodiment, the substrate may be a VOC and the concentration of the exhaled VOC substrate in breath is measured.

Thus, the method uses an exogenous volatile organic compound (EVOC) as tracers of specific in vivo liver-specific metabolic activities. EVOCs can be volatile compounds that, administered to a subject through various routes, undergo metabolism and distribution in the body and are excreted via breath. Additionally, metabolism of EVOCs by liver-specific enzymes can lead to production of other volatile compounds that can also be detected in breath.

In one embodiment, the substrate is a VOC and its metabolite is not a VOC. In another embodiment, the substrate is not a VOC and its metabolite is a VOC. In this case, the concentration of the metabolite in breath is measured. In another embodiment, the substrate is a VOC and its metabolite is a VOC. In this case, the concentration of the substrate and/or the metabolite in breath is measured.

If the substrate is a VOC, it may be labelled or it may not be labelled.

The VOC that is measured according to the methods may not be naturally occurring/produced by the subject and excreted into a biological matrix. This ensures that any readings are not contaminated by endogenous VOCs that are naturally produced and can be found in biological matrices.

In one embodiment, the substrate is a naturally occurring compound (but that is not endogenously produced), for example a food compound. This has the advantage that it can be provided to a subject without the occurrence of side effects. In one embodiment, the substrate does not have any therapeutic benefit. In one embodiment, the substrate is not a non-naturally occurring compound.

In one embodiment, the substrate is a GRAS compound, for example a GRAS compound that is a VOC. “GRAS” is an acronym for the phrase Generally Recognized As Safe. Under sections 201 (s) and 409 of the Federal Food, Drug, and Cosmetic Act, any substance that is intentionally added to food is a food additive, that is subject to premarket review and approval by FDA, unless the substance is generally recognized, among qualified experts, as having been adequately shown to be safe under the conditions of its intended use, or unless the use of the substance is otherwise excepted from the definition of a food additive. For example, the GRAS compound can be a naturally occurring compound. For example, the GRAS compound can be selected from a food or food additive. In one embodiment, the GRAS compound is a vitamin, phenolic flavoring agent, natural oil, alcohol, amino acid or antioxidant. In one embodiment, the GRAS compound is a plant extract. In one embodiment, the GRAS compound is a plant substance primarily used for flavoring, coloring or preserving food. In one embodiment, the GRAS compound is an aliphatic or aromatic terpene hydrocarbon or a terpenoid. In one embodiment, the GRAS compound is an EU approved food flavour.

In one embodiment, said substrate is not a VOC and its metabolite is not a VOC. In that embodiment, the substrate is a labelled reactant and labelled reactant and/or labelled metabolite can be measured in breath. The label may be an isotope label, for example 12C, 13C, 14C, 2H, 14N or 18O.

In an embodiment, the substrate and/or metabolite is a VOC and the substrate is not labelled. Therefore, no labelling is required as the substrate and/or metabolite can be measured in a biological matrix without the use of any labels.

In an embodiment, the enzyme is an AKR enzyme. In one embodiment, the enzyme is AKR1B10. In one embodiment, the substrate is selected from an aldehyde and/or an alcohol. In one embodiment, the enzyme is an AKR enzyme, such as AKR1B10, and the substrate is nonanal and the metabolite nonanol. Where nonanal is used in the present methods, 1-nonanal may be used. 1-nonanal may be metabolised to 1-nonanol and so 1-nonanol may be a metabolite detected in the present methods.

In one embodiment, the enzyme is an AKR enzyme, such as AKR1B10, and the substrate is trans-2-hexenal and the metabolite is trans-2-hexenol.

In one embodiment, the enzyme is an AKR enzyme, such as AKR1B10, and the substrate is hexanal and the metabolite is hexanol.

In one embodiment, the enzyme is an AKR enzyme, such as AKR1B10, and the substrate is benzylaldehyde and the metabolite is benzyl alcohol.

In one embodiment, the enzyme is an AKR enzyme, such as AKR1B10 and the substrate is citral and the metabolite is nerol.

In yet another embodiment, the enzyme used in the method of the invention is a Cytochrome P450 (CYP). CYP are a superfamily of enzymes which, generally, function as monooxygenase.

CYP contains a heme cofactor and are catalysts in steroid hormone synthesis and drug metabolism.

In an embodiment, the enzyme is CYP1A1, CYP1A2, CYP1 B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7 or CYP3A43. In one embodiment, the enzyme is CYP2C19, CYP2C9 and/or CYP3A4

In one embodiment, the enzyme is a CYP enzyme, the substrate is 2-butanone and the metabolite is 3-hydroxy-2-butanone and/or 2,3-butanediol.

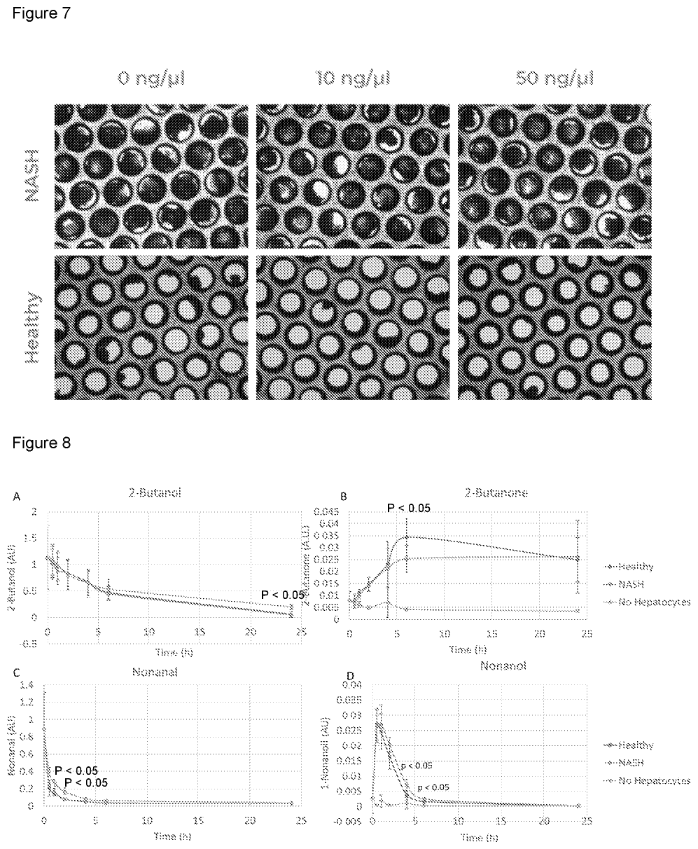

In one embodiment, the enzyme is a CYP enzyme, the substrate is benzoic acid and the metabolite is hippuric acid. Saltzman A, Caraway W T. Cinnamic acid as a test substance in the evaluation of liver function. J Clin Invest 1953; 32 (8): 711-719 demonstrated in FIG. 7 of that publication that benzoic acid is converted to hippuric acid. In one embodiment, the enzyme is glycine N-acyltransferase. In one embodiment, the substrate is benzoic acid and the metabolite is hippuric acid.

In one embodiment, the enzyme is a CYP enzyme, the substrate is 2-pentanone and the metabolite is 2,3-pentanediol.

In one embodiment, a substrate may produce a first metabolite and/or the first metabolite may be used as a substrate to produce a second metabolite. In some embodiments, the concentration of the substrate may be measure. In some embodiments, the concentration of the first metabolite may be measure. In some embodiments, the concentration of the second metabolite may be measured. In some embodiments, the concentration of the substrate and the first metabolite may be measured. In some embodiments, the concentration of the first metabolite and the second metabolite may be measured. In some embodiments, the concentration of the substrate and the second metabolite may be measured. In some embodiments, the concentration of the substrate, the first metabolite, and the second metabolite may be measured.

In one embodiment of the methods above, the concentration of the substrate is measured. In another embodiment of the methods above, the concentration of the metabolite is measured. For example, the concentration of the metabolite nonanol, trans-2-hexenol, hexanol, benzyl alcohol, nerol, 3-hydroxy-2-butanone, 2,3-butanediol, benzoic acid, hippuric acid, 2-pentanone and 2,3-pentanediol is measured. In one embodiment of the methods above, the concentration of the substrate and of the metabolite is measured.

In the methods above, the concentration of one or more substrate and/or one or more metabolite is measured. Thus, in one embodiment, the concentration of multiple metabolites can be measured.

Therefore, the methods provided herein enable the testing of multiple compounds in exhaled breath. This allows testing for the presence of more than one type of disease. Furthermore, multiple compounds which are specific to a certain type of disease can be measured in breath thereby enabling a more accurate diagnosis due to multiple parameters that are assessed. In one embodiment, the invention therefore relates to a method for the detection of a disease comprising assessing the activity of one or more disease-specific enzyme by measuring the concentration of two or more exogenous substrates for said enzyme and/or measuring the concentration of two or more metabolites of said substrate(s) in exhaled breath of a subject.

In one embodiment, the methods for the detection of a liver disease disclosed herein comprise assessing the activity of more than one enzyme by measuring the concentration of two or more exogenous substrates for said enzyme and/or measuring the concentration of two or more metabolites of said substrate(s) in exhaled breath of a subject. The method described herein can therefore be a multiplex method enabling assessment of multiple enzymatic activities simultaneously in the same breath sample(s).

As such, in one aspect, the invention relates to a method for detecting or prognosing NAFLD progressed to the stage of NASH, in a subject, comprising measuring the concentration of an exogenous substrate for a NASH-specific enzyme and/or measuring the concentration of a metabolite of said substrate in a biological sample obtained from said subject wherein said substrate is nonanal. Preferably the invention relates to a method for detecting or prognosing NAFLD progressed to the stage of NASH, in a subject, comprising measuring the concentration of an exogenous substrate for AKR1B10 and/or measuring the concentration of a metabolite of said substrate in a biological sample obtained from said subject wherein said substrate is nonanal. Further nonanal may be metabolised in to nonanol and so in an embodiment the metabolite is nonanol.

In an embodiment, the enzyme is an alcohol dehydrogenase, such as aaADH. In one embodiment, the substrate is selected from an aldehyde and/or an alcohol. In one embodiment, the enzyme is an alcohol dehydrogenase and the substrate is butanol and the metabolite butanone.

In one embodiment, the enzyme is an alcohol dehydrogenase, and the substrate is 2-pentanone and the metabolite is 2-pentanol, 3-hydroxy-2-pentanone and/or 2,3-pentanediol.

In one embodiment, the enzyme is an alcohol dehydrogenase, and the substrate is benzyl alcohol and the metabolites are benzylaldehyde and/or benzoic acid.

In one embodiment, the enzyme is an aldehyde dehydrogenase, and the substrate is benzaldehyde and the metabolite is benzoic acid.

The invention also relates to a method for detecting or prognosing NASH in a subject, comprising measuring the concentration of an exogenous substrate for an enzyme and/or measuring the concentration of a metabolite of said substrate in a biological sample obtained from said subject wherein the substrate is a GRAS compound and wherein the substrate is 2-pentanone and the metabolite is 2-pentanol, 3-hydroxy-2-pentanone and/or 2,3-pentanediol.

As such, in one aspect, the invention relates to a method for detecting or prognosing NAFLD progressed to the stage of NASH, in a subject, comprising measuring the concentration of an exogenous substrate for a NASH-specific enzyme and/or measuring the concentration of a metabolite of said substrate in a biological sample obtained from said subject wherein said substrate is butanol. Preferably, the invention relates to a method for detecting or prognosing NAFLD progressed to the stage of NASH, in a subject, comprising measuring the concentration of an exogenous substrate for and ADH and/or measuring the concentration of a metabolite of said substrate in a biological sample obtained from said subject wherein said substrate is butanol. Further, butanol may be metabolised into butanone and so in an embodiment the metabolite is butanone.

Where butanol is used in the present methods 2-butanol may be used. 2-butanol may be metabolised to 2-butanone and so 2-butanone may be a metabolite detected in the present methods.

In the methods above, the concentration of one or more substrate and/or one or more metabolite is measured. Thus, in one embodiment, the concentration of multiple metabolites can be measured.

By using hepatocyte models as described in the examples which are models for early-stage liver disease the inventors have developed methods that enable the differentiation of NASH from other stages of liver disease and thus enable early diagnosis and intervention. This differentiation relies on alterations of enzymes activity induced by NASH, which results in changes in the metabolic rate of specific compounds. Quantification of these compounds allows identification of subjects with NASH.

This, in another aspect, the invention relates to a method of differentiating between NASH and other stages of NAFLD in a subject, comprising measuring the concentration of an exogenous substrate for an enzyme and/or measuring the concentration of a metabolite of said substrate in a biological sample obtained from a subject wherein the substrate is a GRAS compound.

In another aspect, the invention relates to a method for detecting or prognosing early-stage NASH in a subject, comprising measuring the concentration of an exogenous substrate for an enzyme and/or measuring the concentration of a metabolite of said substrate in a biological sample obtained from said subject wherein the substrate is a GRAS compound.

In another aspect, the invention relates to a method of determining the stage of NASH in a subject, comprising measuring the concentration of an exogenous substrate for an enzyme and/or measuring the concentration of a metabolite of said substrate in a biological sample obtained from a subject wherein the substrate is a GRAS compound.

As explained above, in these methods, the activity or expression of said enzyme is upregulated or downregulated in NASH. For example, the enzyme may be an AKR or an ADH as further explained above. For example, the enzyme may be an AKR and the substrate and metabolite may be selected from those recited above. For example, the substrate may be selected from nonanal, trans-2-hexenal, hexanal, benzylaldehyde, citral, and the corresponding metabolite from nonanol, trans-2-hexenol, hexanol, benzyl alcohol, nerol respectively. For example, the enzyme may be an ADH and the substrate and metabolite may be selected from those recited above. For example, the substrate may be selected from butanol or 2-pentanone and the corresponding metabolite from butanone, 2-pentanol, 3-hydroxy-2-pentanone or 2,3-pentanediol respectively.

In another embodiment, the enzyme is a CYP enzyme. The cytochrome CYP450 (CYP450) enzyme family is responsible for metabolism of most drugs and lipophilic xenobiotics and are therefore of great importance for clinical pharmacology. Although several different families of CYP450 enzymes are present in the human body, the enzymes belonging to 1-, 2-, and 3-families are involved in the metabolism of the great majority of administered therapeutic drugs.

These enzymes transform pro-drugs into corresponding bioactive compounds, as well as active drugs into inactive metabolites that are subsequently excreted from our body. Differences in enzymatic activity of these enzymes lead to different biotransformation of xenobiotics, ultimately resulting in toxicity of a compound or inefficacy of a drug. Many factors contribute to the diversity in CYP450s activity and consequently their metabolic efficiency.

In one embodiment, the CYP450 enzyme is selected from families 1, 2 or 3. For example, the CYP450 enzyme is selected from CYP1A1, CYP1A2, CYP1 B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7 or CYP3A43. In one embodiment, the enzyme is CYP2C19, CYP2C9 and/or CYP3A4. In another embodiment, the liver enzyme is selected from glutathione S-transferase, aryl sulfatase and UDP-glucuronyl transferase or aldehyde dehydrogenases.

In one embodiment, the enzyme is CYP2C19 and/or CYP2C9 and the substrate is limonene. In one embodiment, the liver enzyme is CYP2C19 and/or CYP2C9 and the substrate is limonene and the metabolite is a perillyl alcohol. In one embodiment, the liver enzyme is CYP3A4 and the substrate is eucalyptol.

In one embodiment, the enzyme is glycine N-acyltransferase. In one embodiment, the enzyme is glycine N-acyltransferase, and the substrate is benzoic acid and the metabolite is hippuric acid.

The methods require measuring the concentration of an exogenous substrate and/or a metabolite thereof in a biological sample from a subject, that is a test subject. The term biological sample may be used interchangeably with the term biological matrix. In an embodiment the biological sample or matrix is selected from breath, urine, blood, serum, and/or tissue. The biological sample may be a tissue sample such as adipose tissue, liver, brain, bone marrow, muscle or hair. In an embodiment the biological sample is a sample of bodily fluid. Methods are well known in the art for obtaining bodily fluid samples. In an embodiment the bodily fluid sample may be a sample of blood, urine or exhaled breath. The sample of blood may comprise one or more of blood plasma, red blood cells, white blood cells, platelets. The blood sample may comprise any combination of blood plasma, red blood cells, white blood cells, platelets.

In one embodiment, the sample is exhaled breath.

Where the bodily fluid sample is a sample of exhaled breath, the breath sample can include air exhaled from one or more different parts of the subject's body (e.g. nostrils, pharynx, trachea, bronchioles, alveoli etc.). For the collection of a breath sample and methods of measurement, the device and methods described in WO2017/187120 or WO2017/187141 (both publications are hereby incorporated by reference) can be used.

In embodiments wherein the biological sample is a sample of exhaled breath, this may be obtained by collecting exhaled air from the subject, for example by requesting the subject to exhale air into a gas-sampling container, such as a bag, a bottle or any other suitable gas-sampling product. Preferably the gas-sampling container resists gas permeation both into and out of the bag and/or is chemically inert, thereby assuring sample integrity. Exhaled breath may also be collected using a breath collector apparatus. Preferably, collection of a sample of exhaled breath is performed in a minimally invasive or a non-invasive manner.

The determination of the amount of one or more VOCS in a sample of exhaled breath from a subject may be performed by the use of at least one technique including, but not limited to, Gas-Chromatography (GC), Gas-Chromatography-lined Mass Spectrometry (GC/MS), Liquid Chromatography-tandem mass spectrometry (LC/MS), Ion Mobility Spectrometry/Mass Spectrometry (IMS/MS), Proton Transfer Reaction Mass-Spectrometry (PTR-MS), Electronic Nose device, quartz crystal microbalance or chemically sensitive sensors.

The amount of one or more VOCs in a sample of exhaled breath from a subject may be determined using thermal desorption-gas chromatography-time of flight-mass spectrometry (GC-fof-MS). In certain embodiments, breath of the subject is collected in an inert bag, then the content of the bag is transported under standardised conditions onto desorption tubes and VOCs are analyzed by thermally desorbing the content of the tube and then separated by capillary gas chromatography. Then volatile organic peaks are detected with MS and identified using for example a library, such as the National Institute of Standards and Technology. Thermal desorption may be performed at the GC inlet at a temperature of, e.g., about 200-350° C. In all chromatography, separation occurs when the sample mixture is introduced (injected) into a mobile phase. Gas chromatography (GC) typically uses an inert gas such as helium as the mobile phase. GC/MS allows for the separation, identification and/or quantification of individual components from a biological sample. MS methods which may be used with the present invention include, but are not limited to, electron ionization, electrospray ionization, glow discharge, field desorption (FD), fast atom bombardment (FAB), thermospray, desorption/ionization on silicon (DIOS), Direct Analysis in Real Time (DART), atmospheric pressure chemical ionization (APCI), secondary ion mass spectrometry (SIMS), spark ionization and thermal ionization (TIMS). Matrix assisted laser desorption ionization time-of-flight mass spectrometry (MALDI-TOF-MS) is an example of a mass spectroscopy method which may be used to determine one or more VOCs from a sample of exhaled breath from a subject.

In one embodiment, the method comprises collecting different selected exhaled breath samples, or fractions thereof, on a single breath sample capture device, the method comprising the steps of:
(a) collecting a first exhaled breath sample by contacting the sample with a capture device comprising an adsorbent material;
(b) collecting a second exhaled breath sample by contacting the second sample with said capture device, wherein the first and second exhaled breath samples are caused to be captured on the capture device in a spatially separated manner.

Thus, the invention also relates to a method comprising
(a) administering a substrate as described herein
(b) collecting a first exhaled breath sample by contacting the sample with a capture device comprising an adsorbent material;
(c) collecting a second exhaled breath sample by contacting the second sample with said capture device, wherein the first and second exhaled breath samples are caused to be captured on the capture device in a spatially separated manner.

In some embodiments, the capture device comprises an adsorbent material in the form of a porous polymeric resin. Suitable adsorbent materials include Tenax® resins and Carbograph® materials. Tenax® is a porous polymeric resin based on a 2,6-diphenyl-p-propylene oxide monomer. Carbograph® materials are graphitized carbon blacks. In one embodiment, the material is Tenax GR, which comprises a mixture of Tenax® TA and 30% graphite. One Carbograph® adsorbent is Carbograph 5TD. In one embodiment, the capture device comprises both Tenax GR and Carbograph 5TD. The capture device is conveniently a sorbent tube. These are hollow metal cylinders, typically of standard dimensions (3½ inches in length with a ¼ inch internal diameter) packed with a suitable adsorbent material.

A non-NASH subject is a subject that does not have NASH but may or may not have NAFL. A non-NASH subject also includes a healthy subject. As used herein, “healthy subject” is defined as a subject, i.e. a human, that does not have the NASH disease of interest.

As used herein, “reference value” means a value determined by performing the testing method on a plurality of reference subjects. A reference subject can be a healthy subject or a subject diagnosed with a disease.

A “likelihood of a disease state” means that the probability that the disease state exists in the subject specimen is about 50% or more, for example 60%, 70%, 80% or 90%.

A decrease or increase as used herein can be 5%, 10%, 20%, 30%, 40%, 50% or more or at least or about 2 fold, at least or about 2.5 fold, at least or about 3 fold, at least or about 3.5 fold, at least or about 4 fold, at least or about 5 fold, at least or about 6 folds, at least or about 7 fold, at least or about 8 fold, at least or about 9 fold or at least or about 10 fold.

The methods of the present invention involve determining the concentration of the substrate and/or metabolite in the breath sample of the test subject and then comparing the concentration to a reference/baseline value or range. Typically, the reference/baseline value is representative of the concentration of the substrate and/or metabolite in a healthy person or non-NASH subject not suffering from or destined to develop NASH.

The sample may be obtained at any time point. The sample may be obtained at any time point before or after the administration of the substrate(s), such as about 10 minutes, about 30 minutes, about 1 hour, about 2 hours, about 3 hours, about 4 hours, about 5 hours, about 6 hours, about 7 hours, about 8 hours, about 10 hours, about 12 hours, about 15 hours, about 18 hours, about 20 hours, about 22 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 3 years, about 5 years or longer before or after the start of the treatment or therapy. The time point may also be earlier or later.

Variation of levels of substrate and/or metabolite from the baseline/reference value or range (either up or down) indicates that the patient has NASH, an increased risk of NASH and/or an increased risk of long-term mortality. For example, in embodiments where the enzyme is an AKR, an increased concentration of the metabolite in exhaled breath compared to a baseline value in a healthy individual indicates a risk of NASH. AKR enzymes are overexpressed in NASH disease and conversion of the substrate to the metabolite is therefore increased. Conversely, a decreased level of the substrate in exhaled breath indicates a risk of NASH.

For example, in embodiments where the enzyme is an ADH, healthy subjects have higher production of 2-butanone than NASH-patients, as the alcohol dehydrogenase pathway involved in the conversion of 2-butanol to 2-butanone is downregulated in NASH.

The algorithm used to calculate a risk assessment score in a method disclosed herein may group the concentration values of the substrate and/or metabolite, and the risk score can be derived from any algorithm known in the art. The algorithms are sets of rules for describing the risk assessment of NASH. The rule set may be defined exclusively algebraically but may also include alternative or multiple decision points requiring domain-specific knowledge, expert interpretation or other clinical indicators. Many algorithms that can provide different risk assessments can be developed using concentration profiles of a suitable substrate and/or metabolite. For example, the risk scores of an individual may be generated using a Cox proportional hazard model. An individual's prognostic categorization can also be determined by using a statistical model or a machine learning algorithm, which computes the probability of recurrence based on the individual's concentration of the substrate and/or metabolite.

Based on the determination of a risk, individuals can be partitioned into risk groups (e.g., tertiles or quartiles) based on a selected value of the risk score, where all individuals with values in a given range can be classified as belonging to a particular risk group. Thus, the values chosen will define risk groups of patients with respectively greater or lesser risk. Risk groups can further be classified on different ranges of mortality, for example, on 6 month, 1-year, 2-year, 3-year, 4-year, 5-year, 10-year, 25-year mortality. Risk groups can further be classified on different ranges of events associated with NASH, which can include, but is not limited, likelihood of progression to NASH with fibrosis or NASH with cirrhosis.

The concentration of the substrate and/or metabolite can be measured using methods known in the art. The concentration as used herein means the content or mass of the substrate and/or metabolite in the biological sample as expressed, for example in grams/litre (g/l). In one embodiment, concentration is measured over time, for example by measuring the kinetics of the clearance. For example, concentration is measured by assessing the kinetic profile of the clearance of the substrate for example from breath which is then used as a readout. In addition or alternatively, secretion of metabolic products that can derive from the substrate can be measured over time. For example, clearance of the substrate from biological sample and secretion of metabolic products can both be measured in the same biological sample at the same time or at different times.

In one embodiment, the concentration or amount of the substrate and/or its metabolite may be determined in absolute or relative terms in multiple biological samples, e.g. in a first breath sample (collected at a first time period) and in a second and/or further breath sample (collected at a later, second or further time period), thus permitting analysis of the kinetics or rate of change of concentration thereof over time.

In some embodiments, where the biological sample is an exhaled breath sample the capture device comprises an adsorbent material in the form of a porous polymeric resin. Suitable adsorbent materials include Tenax® resins and Carbograph® materials. Tenax® is a porous polymeric resin based on a 2,6-diphenyl-p-propylene oxide monomer. Carbograph® materials are graphitized carbon blacks. In one embodiment, the material is Tenax GR, which comprises a mixture of Tenax® TA and 30% graphite. One Carbograph® adsorbent is Carbograph 5TD. In one embodiment, the capture device comprises both Tenax GR and Carbograph 5TD. The capture device is conveniently a sorbent tube. These are hollow metal cylinders, typically of standard dimensions (3½ inches in length with a ¼ inch internal diameter) packed with a suitable adsorbent material.

In one embodiment, the methods of the invention further comprise establishing a test subject value for one or more substrate and/or metabolite concentration. Said test subject value may be compared to one or more reference (control) values wherein a difference in the test subject value and a reference value indicates a likelihood of NASH.

In one embodiment, said reference value is from non-NASH subjects, e.g. healthy subjects.

In another embodiment, the reference value is from subjects diagnosed with NASH (with or without fibrosis), or a subject where NASH has progressed to cirrhosis or HCC.

Reference levels the test compound (i.e. substrate or metabolite) can be determined by determining the level of the test compound in a sufficiently large number of samples obtained from normal, healthy control subjects to obtain a pre-determined reference or threshold value. A reference level can also be determined by determining the level of the test compound in a sample from a patient prior to treatment.

In one embodiment, the methods of the invention further comprise comparing the subject value to one or more reference value. In one embodiment, said reference value is from non-NASH subjects, e.g. healthy subjects. In another embodiment, the reference value is from subjects diagnosed with NASH. In another embodiment the reference value is the value of a subject that has been diagnosed with non-alcoholic fatty liver (NAFL). In another embodiment said reference value is the value of a subject that has progressed to cirrhosis or HCC.

In one embodiment, the reference value is a NAFL subject value corresponding to values calculated from NAFL subjects. In one embodiment, the presence of one or more subject values at quantities greater than their respective range of healthy subject values indicates a substantial likelihood of a NASH disease state in the test subject.

In one embodiment, the reference value is a healthy subject value corresponding to values calculated from healthy subjects. In one embodiment, the presence of one or more subject values at quantities greater than their respective range of healthy subject values indicates a substantial likelihood of a NASH disease state in the test subject.

In one embodiment, an increased concentration of the test compound, e.g. of the substrate or metabolite, e.g. 5%, 10%, 20%, 30%, 40%, 50% or more, compared to the reference value indicates a diagnosis of NASH or a risk that the subject will develop NASH.

In one embodiment, a decreased concentration of the test compound, e.g. of the substrate or metabolite, e.g. 5%, 10%, 20%, 30%, 40%, 50% or more, compared to the reference value indicates a diagnosis of NASH or a risk that the subject will develop NASH.

In one embodiment, when an appropriate reference is indicative of a subject being free of NASH a detectable difference (e.g., a statistically significant difference) between the value determined from a subject in need of characterization or diagnosis of NASH and the appropriate reference may be indicative of NASH in the subject. In one embodiment, when an appropriate reference is indicative of NASH, a lack of a detectable difference (e.g., lack of a statistically significant difference) between the value determined from a subject in need of characterization or diagnosis of NASH and the appropriate reference may be indicative of NASH in the subject. In an embodiment, an increase in the concentration of the metabolite compared to the reference sample is indicative of NASH.

Thus, in one aspect, the methods include detecting the concentration of the substrate and/or metabolite in exhaled breath from the subject and diagnosing the subject as having a likelihood or increased risk of a NASH disease state if the level of one or more of the substrate and/or metabolite is different from the healthy reference subject value.

Thus, any of the methods as described herein may further comprise the steps of:
a) Comparing the amount of one or more VOC in a biological sample with a reference value, said reference value representing a known diagnosis, prognosis and/or monitoring status of NASH;
b) Finding a deviation or no deviation of the amount of said one or more VOC from said reference value; and
c) Attributing said finding of deviation or no deviation to a particular diagnosis, prognosis and/or monitoring status of NASH, in the subject.

The term “deviation of the amount” refers either to elevated or reduced amounts of one or more VOC in a biological sample from a subject compared to a reference value. By “elevated amounts” we mean that the amount of said one or more VOCS in a biological sample from a subject is statistically higher than the reference value. By “reduced amounts” we mean that the amount of said one or more VOC in a biological sample from a subject is statistically lower than the reference value. The amount may be considered to be statistically higher or lower if its value differs from a predetermined threshold value. This threshold value can, for example, be the median of the amount of VOC determined in a biological sample from a population of healthy subjects.

The term “no deviation of the amount” refers to similar or unchanged amounts of one or more VOC of the invention in a sample of exhaled breath from a subject compared to a reference value. By “similar or unchanged level” is meant that the difference of the amount of said one or more VOC in a biological sample from the subject compared to the reference value is not statistically significant. Preferably, the reference value is obtained in samples of exhaled breath obtained from one or more subjects of the same species and the same sex and age group as the subject in which NASH is to be determined, prognosed or monitored. Alternatively, the reference value may be a previous value for the amount of one or more VOCS obtained in a sample of exhaled breath from a specific subject. This kind of reference value may be used if the method is to be used for monitoring the NASH, e.g. over time, or to monitor the response of a subject to a particular treatment.

The method may also comprise determining a risk score of the subject based on the concentration of the metabolite and/or substrate in the sample and using the risk score to provide a prognosis for the subject, wherein the risk score is indicative of said prognosis.

The methods may comprise determining the concentration of two or more exogenous substrates for a NASH specific enzyme and/or determining the concentration of two or more metabolites of said substrates.

In the methods of the invention the subject has been administered the exogenous substrate for a NASH specific enzyme. In certain embodiments the method may comprises a step of administering the exogenous substrate for a NASH specific enzyme to a subject. Administration of the substrate may be performed via any reasonable route including but not limited to oral, parenteral, sublingual, rectal, vaginal, ocular, intranasal, pulmonary, intradermal, intravitrial, intramuscular, intraperitoneal, intravenous, subcutaneous, intracerebral, transdermal, transmucosal. Parenteral administration includes, for example, intravenous, intramuscular, intraarterial, intraperitoneal, intranasal, rectal, intravesical, intradermal, topical or subcutaneous administration. Preferably the substrate is administered orally. Once the subject has been administered the substrate the concentration of the substrate and/or metabolites thereof can be determined in biological samples obtained from said subject.

An aspect relates to a method of monitoring the progression of NASH in a subject, comprising measuring the concentration of an exogenous substrate for a NASH specific enzyme and/or measuring the concentration of a metabolite of said substrate in a biological sample obtained from a subject wherein said substrate and/or its metabolite is a VOC and wherein the substrate is GRAS compound and wherein the enzyme is an aldo-ketoreductase (AKR) or an alcohol dehydrogenase as described above.

An aspect relates to a method for determining efficacy of a treatment comprising in a subject diagnosed with NASH, assessing the activity of an enzyme by measuring the concentration of an exogenous substrate for said enzyme and/or measuring the concentration of a metabolite of said substrate in a biological sample obtained from the subject, wherein said subject has received treatment for NASH and wherein the enzyme is an aldo-ketoreductase (AKR) or an alcohol dehydrogenase as described above.

The methods for determining efficacy of a treatment may contain the following steps: (a) obtaining a first sample from the patient before initiation of the treatment or therapy (or at a first time point after initiation of the treatment or therapy, or when the treatment or therapy is initiated); (b) assaying the level of the substrate and/or metabolite in the first sample; (c) obtaining a second sample from the patient after initiation of the treatment or therapy (or at a second time point after initiation of the treatment or therapy); (d) assaying the level of the I the substrate and/or metabolite in the first sample and (e) comparing of the substrate and/or metabolite level in the first sample with of the substrate and/or metabolite level in the second sample.

Depending on the of the substrate and/or metabolite assayed, if the level increases or decreases compared to the level of the substrate and/or metabolite obtained in the first sample, the therapy is considered to be effective. An effective treatment or therapy may be continued, or discontinued if the patient's condition has improved and is no longer in need of treatment. An ineffective treatment may be altered or modified, or replaced with other treatment.

The treatment may comprise surgery or at least one drug selected from statins, incretin analogues, metformin, rimonabant, thiazolidinediones, and orlistat.

In order to determine the efficacy of treatment multiple samples may be obtained at various times points. As such the method may comprise analysing a first biological sample obtained from said subject at a first time point, and then analysing one or more additional biological samples obtained from said subject at one or more additional time points or ratios thereof. Thus, the method may also comprise the step of administering a treatment.

In an embodiment said treatment of NASH is gastric bypass surgery, and/or a drug-based treatment comprising the administration of at least one drug selected from statins, incretin analogues, metformin, rimonabant, thiazolidinediones, and orlistat. Treatments of NASH are known in the art, se Ganguli et al Hepat Med. 2019; 11:159-178.

In one embodiment of any of the methods described, the substrate is not limonene. In one embodiment of any of the methods described, the substrate is not a CYP enzyme.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. Therefore, certain embodiments employ processes involving data stored in or transferred through one or more computer systems or other processing systems. Embodiments also relate to apparatus for performing these operations. This apparatus can be specially constructed for the required purposes, or it can be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel.

In some embodiments, a microprocessor is part of a system for determining the presence of one or more mRNA or miRNA associated with a liver disease; generating standard curves; determining a specificity and/or sensitivity of an assay or marker; calculating an ROC curve; sequence analysis; all as described herein or is known in the art.

In some embodiments, a microprocessor is part of a system for determining the amount, such as concentration, of one or more substrate and or metabolite associated with NASH; generating standard curves; determining a specificity and/or sensitivity of an assay or marker; calculating an ROC curve; sequence analysis; all as described herein or is known in the art. The amount of one or more substrates and or metabolites can be determined by abundance, measured per mole or millimole.

The amount of one or more substrate and or metabolite can be determined by assays known to the skilled person and described herein, including measurements using an optical signal or other measurement known to one of skill.

In some embodiments, a microprocessor or computer uses an algorithm to measure the amount of one or more substrate and or metabolite. The algorithm can include a mathematical interaction between a marker measurement or a mathematical transform of a marker measurement. The mathematical interaction and/or mathematical transform can be presented in a linear, nonlinear, discontinuous or discrete manner.

In some embodiments, a software or hardware component receives the results of multiple assays and determines a single value result to report to a user that indicates a NASH disease risk based on the results of the multiple assays. Related embodiments calculate a risk factor based on a mathematical combination (e.g., a weighted combination, a linear combination) of the results from multiple assays as described elsewhere herein.

### Compositions and Kits

The invention relates to a kit for the detection, diagnosis, screening or prognosis of NASH, differentiating NASH from NAFL or for determining efficacy of a NASH treatment comprising nonanal and/or butanol and a device for capturing a biological sample from a patient.

The kit as described herein may include a composition for administration that comprises the substrate i.e. nonanal, butanol, trans-2-hexenal, hexanal, benzylaldehyde, citral, or 2-pentanone or limonene. This may be formulated as an oral administration, e.g. as a tablet or capsule.

The kit may comprise instructions for evaluating or monitoring NASH in a patient based on the level of the substrate and/or metabolite of interest. In some embodiments, the kit contains reagents for measuring the level of substrate and/or metabolite of interest.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed (e.g., sterile, pharmaceutically acceptable buffer and/or other diluents). However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

This component of the kit be for administration as described above. It may also include a pharmaceutically acceptable carrier or vehicle. This can be a particulate, so that the compositions are, for example, in tablet or powder form. The term “carrier” refers to a diluent, adjuvant or excipient, with which a substrate is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. The composition can be in the form of a liquid, e.g., a solution, emulsion or suspension. The liquid can be useful for delivery by injection, infusion (e.g., IV infusion) or sub-cutaneously. As a solid composition for oral administration, the composition can be formulated into a powder, granule, compressed tablet, pill, capsule, chewing gum, wafer or the like form. Compositions can take the form of one or more dosage units.

The substrate can be contained in a composition, such as a nutritional supplement. The different absorption rates of the substrate into the blood can cause significant shifts in the time of maximum concentration on breath. Therefore, in one embodiment, the substrate is provided in a formulation to ensure fast delivery. In one embodiment, the substrate is formulated as a liquid. In another embodiment, the substrate is formulated as a fast release/fast dissolving tablet or capsule. This ensures that the absorption has a much shorter time constant compared to the washout.

In another embodiment, the subject is fasting overnight and fasting can be combined with the provision of the substrate as a liquid or fast release/dissolving tablet or fast release/dissolving capsule or other oral administration format.

Typically, the amount of the substrate administered as part of the methods of the invention or the amount of the substrate included in the composition comprised in the kit is at least about 0.01% of the substrate by weight of the composition. When intended for oral administration, this amount can be varied to range from about 0.1% to about 80% by weight of the composition. For administration by injection, the composition can comprise from about typically about 0.1 mg/kg to about 250 mg/kg of the subject's body weight, preferably, between about 0.1 mg/kg and about 20 mg/kg of the subject's body weight, and more preferably from about 1 mg/kg to about 10 mg/kg of the subject's body weight.

### Uses

The invention also relates to the use of an exogenous substrate for an enzyme in any of the method described herein. In an embodiment the exogenous substrate is selected from nonanal, butanol, trans-2-hexenal, hexanal, benzylaldehyde, citral and/or 2-pentanone.

The invention also relates to nonanal, butanol, trans-2-hexenal, hexanal, benzaldehyde, citral, 2-pentanone, nonanol, butanone trans-2-hexenol, hexanol, benzyl alcohol, nerol, 2-pentanol, benzoic acid, hippuric acid, 2,3-butanediol, 3-hydroxy-2-pentanone and/or 2,3-pentanediol thereof for use in a method of detecting, staging, monitoring or prognosing NASH in a subject, comprising measuring the concentration of one or more of nonanal, butanol, or a metabolite thereof in a biological sample obtained from said subject. The invention also relates to nonanal, butanol, trans-2-hexenal, hexanal, benzaldehyde, citral, 2-pentanone, nonanol, butanone trans-2-hexenol, hexanol, benzyl alcohol, nerol, 2-pentanol, benzoic acid, hippuric acid, 2,3-butanediol, 3-hydroxy-2-pentanone and/or 2,3-pentanediol may be for use in an in vivo, in vitro or ex vivo method of detecting, screening, monitoring, diagnosing or prognosing NASH in a subject.

An aspect of the invention relates to the use of nonanal, butanol, trans-2-hexenal, hexanal, benzaldehyde, citral, 2-pentanone, nonanol, butanone trans-2-hexenol, hexanol, benzyl alcohol, nerol, 2-pentanol, benzoic acid, hippuric acid, 2,3-butanediol, 3-hydroxy-2-pentanone and/or 2,3-pentanediol as a biomarker for NASH disease. Also within the scope of the invention is the use of nonanal, butanol, trans-2-hexenal, hexanal, benzaldehyde, citral, 2-pentanone, nonanol, butanone trans-2-hexenol, hexanol, benzyl alcohol, nerol, 2-pentanol, 3-hydroxy-2-pentanone and/or 2,3-pentanediol thereof as a biomarker of one or more of NASH without fibrosis, NASH with fibrosis, or NASH-cirrhosis.

The invention also relates to nonanal, butanol, trans-2-hexenal, hexanal, benzaldehyde, citral, 2-pentanone, nonanol, butanone trans-2-hexenol, hexanol, benzyl alcohol, nerol, 2-pentanol, limonene, benzoic acid, hippuric acid, 2,3-butanediol, 3-hydroxy-2-pentanone,2,3-pentanediol and/or a perillyl alcohol thereof for use in a method of differentiating NAFL and NASH in a subject, comprising measuring the concentration of one or more of nonanal, butanol, trans-2-hexenal, hexanal, benzaldehyde, citral, 2-pentanone, nonanol, butanone trans-2-hexenol, hexanol, benzyl alcohol, nerol, 2-pentanol, limonene, benzoic acid, hippuric acid, 2,3-butanediol, 3-hydroxy-2-pentanone,2,3-pentanediol and/or a perillyl alcohol in a biological sample obtained from said subject.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further illustrated in the following non-binding examples.

### EXAMPLES

### Example 1 Identification of Genes Differentially Expressed in NASH Vs Healthy/NAFLD Tissue

Gene expression datasets were obtained from dedicated repositories. These datasets were generated from liver tissues obtained from healthy subjects, or subjects affected by NAFLD at stages ranging from simple steatosis to NASH to fibrosis. Analysis for genes differentially expressed between subjects with NASH against those with simple steatosis or healthy, identified AKR1B10 as gene upregulated in NASH at the transcriptomic level. Additional exploration of available literature showed that AKR1B10 increases in NASH also at the protein level.

### Example 2 Bioproduct Production in Cell-Based Assays

Identified substrates for AKR1B10 are nonanal, trans-2-hexenal, hexanal, benzylaldehyde, citral. We have tested conversion of these substrates to the respective bioproduct 1-nonanol, trans-2-hexenol, hexenol, benzyl alcohol, and nerol, in a cancer cell line overexpressing AKR1B10. Addition of the substrates to the culture media results in the conversion to the bioproducts in a timeframe of 6 hours. Ablation of AKR1B10 or treatment of the cells with AKRs inhibitors, results in reduced or absent bioproduct generation.

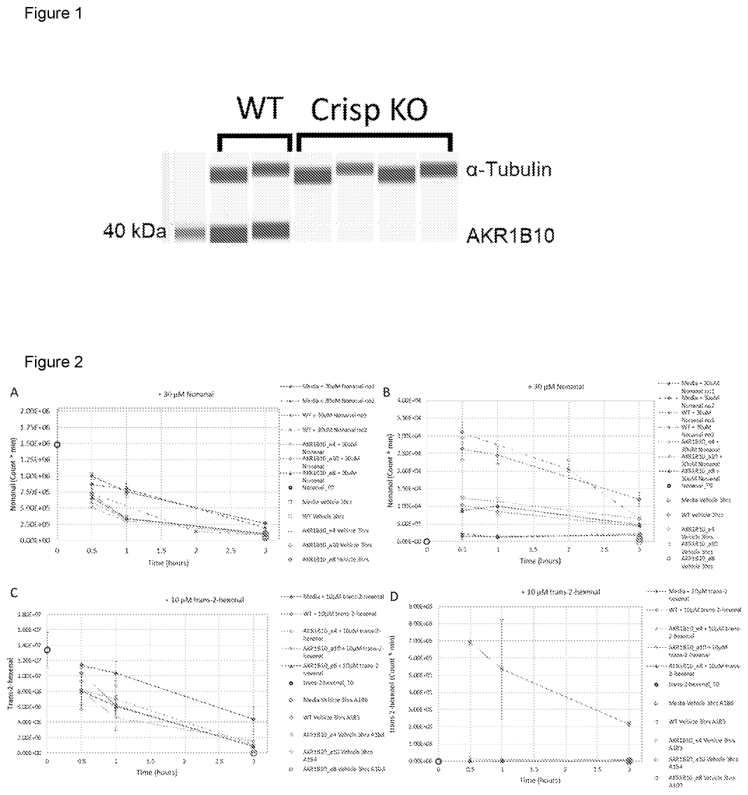

A549 lung cancer cells were targeted with a CRISP/Cas system to ablate the protein expression of AKR1B10. Specific sgRNA for the AKR1B10 gene were co-transfected in cells with SpCas9 using ribonucleoproteins (RNPs). Clonal cells were generated by using limiting dilution. After expansion, clones were screened using PCR, a total of 4 correctly edited clones were identified. Resulting protein extract for WT and AKR1B10 KO cells were analysed using the Jess system and samples were probed with an AKR1B10 specific antibody, and with an antibody against α-Tubulin as independent reporter. Western blot analysis of cell extracts obtained from wild type (WT) or AKR1B10 CRISP/Cas knock out (KO), 4 different clones were generated. Lack of antibody staining for AKR1B10 in KO cells lysates indicates complete and constitutive ablation of this enzyme. The results are shown in FIG. 1.

5×10⁴ cells/mL of WT or 3 AKR1B10 KO clone cells were seeded in 24-well plates using cell culture media and let adhere and proliferate for 48 hours to confluence. Then, they were treated with 30 μM nonanal or 10 UM trans-2-hexenal. Aliquots of media were collected at 0.5, 1, and 3 hours post treatment. Headspace analysis was performed by using Centri, coupled with a GC-MS Orbi-trap system. Peak identification of nonanal, 1-nonanol, trans-2-hexenal, and trans-2-hexenol, was performed by comparing the peak against reference standards. The results are shown in FIG. 2.

Medium supplemented with 30 μM nonanal and incubated for 3 hours shows reduction of nonanal due to spontaneous evaporation. Addition of the medium supplemented with 30 UM nonanal to WT or AKR1B10 KO cell lines shows an increased reduction over 3 hours indicating that cells are metabolizing nonanal (FIG. 2A). In all the conditions, nonanal is not detected if only methanol was added to the culture media. In FIG. 2B, the same medium was used in FIG. 2A, supplemented with 30 UM nonanal. This shows no production of 1-nonanol in the absence of cells. Addition of the medium to the WT or KO cells shows production of 1-nonanol with levels that are 3 times higher in the WT compared to the KO cells. No 1-nonanol is detected if nonanal is not added to the medium in all the conditions.

Medium supplemented with 10 UM trans-2-hexenal and incubated for 3 hours shows reduction of trans-2-hexenal due to spontaneous evaporation. Addition of the medium supplemented with 10 UM trans-2-hexenal to WT or AKR1B10 KO cell lines shows an increased reduction over 3 hours indicating that cells are metabolizing trans-2-hexenal. In all the conditions, trans-2-hexenal is not detected if only methanol was added to the culture media. FIG. 2D. The same medium used in FIG. 2C, supplemented with 10 UM trans-2-hexenal shows no production of trans-2-hexenol in the absence of cells. Addition of the medium to WT or KO cells shows production of trans-2-hexenal with levels that are 10 times higher in the WT compared to the KO cells. No trans-2-hexenol is detected if trans-2-hexenal is not added to the medium in all the conditions.

results indicate that nonanal and trans-2-hexenal are substrates for AKR1B10 and they are converted to respectively 1-nonanol and trans-2-hexenol by this enzyme.

### Example 3. Nonanal and 2-Butanol Washout Experiments

An analysis of nonanal and 2-butanol washout of breath samples collected from three volunteers investigated before and after the intake of 500 mg of Nonanal and 100 mg of 2-butanol was performed. Three breath collections have been performed per each volunteer. The area of nonanal and 2-butanol, and their respective bioproducts nonanol and 2-butanone in collected breath samples have shown spikes in intensities within 20 minutes for the three volunteers.

Three Volunteers participated in this trial for 3 times. They swallowed an emulsion containing 500 mg of Nonanal and 100 mg of 2-butanol and their breath was collected at 8 different time points before and after the emulsion intake using the ReCIVA, a face mask containing tubes for breath collection, and storage of exhaled VOCs.

Appearance on breath and conversion of the probes to their bioproducts was measured using gas-chromatography mass-spectrometry (GC-MS).

Nonanal and 2-butanol were detected in the breath samples for the three volunteers collected after the emulsion intake. The levels of nonanal resulted increased within 20 minutes. Nonanol showed the same trend of Nonanal with a spike within 20 minutes after the nonanal intake. The levels of 2-butanol resulted increased within 20 minutes. 2-butanone showed the same trend of 2-butanol with a spike within 20 minutes after the 2-butanol intake

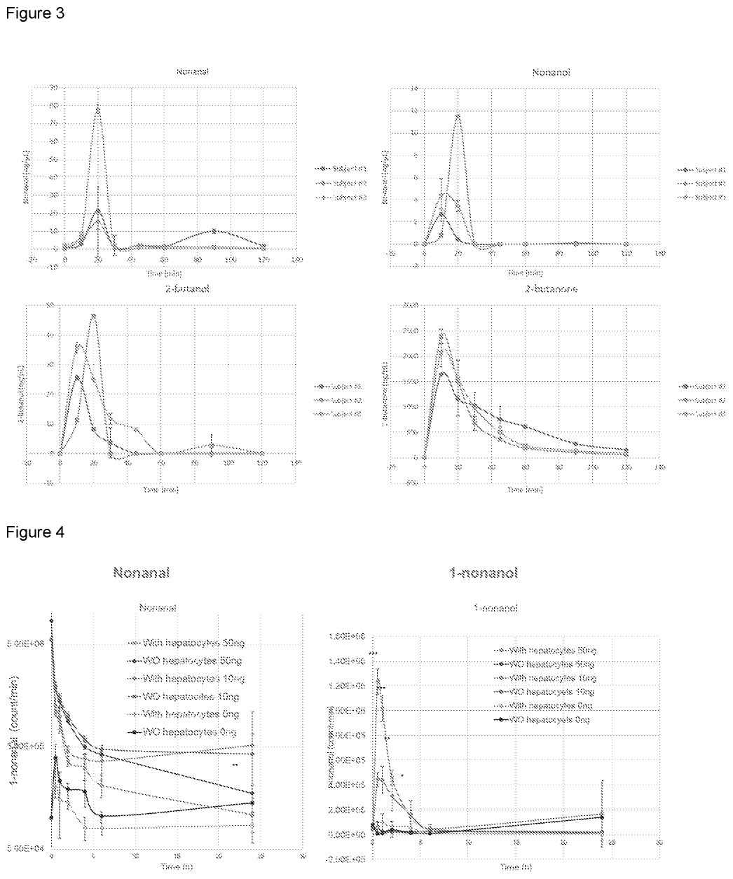

The washout is shown in FIG. 3.

### Example 4. Detection of VOCs from Healthy Human Hepatocytes

### Aim

To investigate the metabolism of 2-butanol and nonanal by healthy human hepatocytes cells over 24 hours and compare it to changes in the concentration of these compounds on the control plate without any cells over the same period of time.

### Objectives

To understand the metabolism of 2-butanol and nonanal in primary hepatocytes.
To assess the change of the levels of these compounds in culture media with hepatocytes compared culture media without hepatocytes over 24 hours from spiking and compare them.
Assess the rates of metabolic breakdown of the specified VOCs by healthy hepatocytes

### Methods

Organ-on-a chip samples were used.

Organ-on-a chip is a three-dimensional hepatocytes culturing system developed by a company called CN-Bio Ltd. Aliquots of media spiked with VOCs and collected at different time points are analysed using CENTRI-GC-MS a system for VOCs headspace detection and quantification. The experiment contained samples from healthy hepatocytes in the culture medium exposed to 2-butanol, nonanal at one of the concentrations: 50, 10, 2 and 0 ng/μL, and the control which is the culture medium without hepatocytes containing 2-butanol, and nonanal of the same concentration.

System suitability test (SST) and 2 standards check (0, 2, 10 and 50 ng/μL), were run in duplicate at the beginning, in the middle and at the end of the sequence to mitigate against the risk of an instrumental deviation.

Empty tubes were also run at the beginning, in the middle and at the end of the sequence to mitigate against the risk of a carryover 0.5 μL Internal standards (1000 ng/μL) were spiked into each sample prior to analysis.

### Conclusions

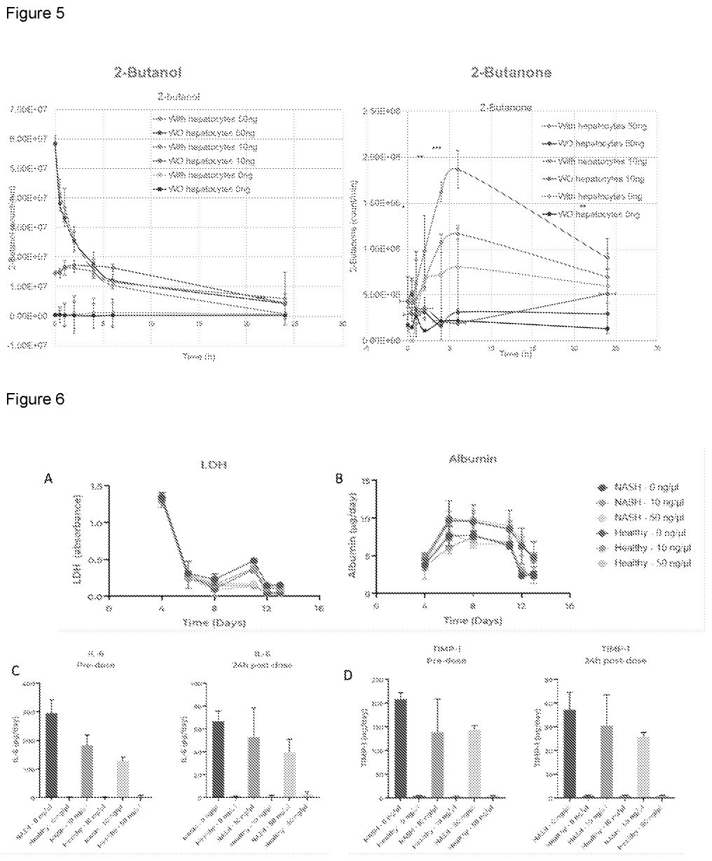

Sample from a plate containing media with hepatocytes and only media treated with 2-butanol, and nonanal were analysed. 50, 10, 2 and 0 ng/μL of eVOC was used. Instrumental performance check was performed and showed a good consistence throughout the sequence. 2-butanol, and nonanal showed a decrease both in media alone and media with hepatocytes. The rate of metabolism between were observed in all compounds when exposed to culture media with hepatocyte and culture media only 1-nonanol (bioproduct of nonanal) and 2-butanone (bioproduct of 2-butanol) showed a spike and subsequent decrease, which corresponds to metabolism of nonanal and 2-butanol. This is shown in FIGS. 4 and 5.

### Example 5. Detection of VOCs from Healthy Human Hepatocytes and Diseased Human Hepatocytes

Organ-on-a-chip containing healthy human hepatocytes and human hepatocytes treated to generate a model of a healthy or NASH liver disease were used in the following experiments. The Organ-on-a-chip containing healthy human hepatocytes are similar to the Organ-on-a-chip described above, but the experiment also included a model of NASH hepatocytes. Organ-on-a-chip described above, but the experiment also included a model of NASH hepatocytes. The latter are obtained by treating healthy hepatocytes with Kupfer cells and hepatocytes stellate cells. These additional cells induce inflammation and fibrosis accumulation at a level that resembles those observed in the NASH liver. This model has been validated by gene expression and protein expression and showed profiles that align with those observed in the NASH liver biopsied from affected subjects.

As explained in more detail below, healthy and NASH hepatocytes showed differential metabolism of nonanal and 2-butanol. NASH hepatocytes, which overexpress AKR1B10, showed higher production of 1-nonanol, when treated with nonanal, compared to healthy hepatocytes. Conversely, NASH hepatocytes showed reduced production of 2-butanone when treated with 2-butanol, as expected, given downregulation of the alcohol dehydrogenase pathway reported in NASH.

Primary human hepatocytes (PHHs), Kupffers cells (KCs), and hepatic stellate cells (HSCs) were seeded onto CN-Bio's PhysioMimix LC12 MPS culture plates at 6×10⁵ cells for PHHs and 6×10⁴ cells for HKCs and HSCs (necessary to induce the NASH phenotype) per well in CN-Bio's seeding medium. Throughout the experiment the cells were maintained at a medium flow rate of 1 μl/s. At days 4, 6, 8, 11, and 12 post seeding, medium aliquots were taken to measure LDH and albumin to assess cell health. At day 13 (24 hours post VOCs treatment), final medium collection for analysis was performed. Scaffolds were fixed in 4% formaldehyde for Oil Red O staining to assess lipid accumulation.

Lactate dehydrogenase level (LDH) was assessed using the CytoTox 96 Cytotoxicity (LDH) Assay Kit. Albumin was measured using the AssayMax Albumin ELISA Kit. IL-6 and TIMP-1 ELISAs (R&D DuoSet kits) were performed on medium samples collected at day 11 and 13 (24-hour post dosing) to assess levels of inflammation and fibrosis. The results are shown in FIG. 6.

Lactate dehydrogenase level (LDH) was measured in the supernatant of healthy and NASH hepatocytes, showing that levels reduce over days of culturing, indicating that the cell stress due to culture procedure is not present at the time when the cells are treated with nonanal and 2-butanol (6A). Levels of albumin in the supernatant of hepatocytes showing that NASH hepatocytes produce less albumin than healthy hepatocytes as expected given the disease phenotype observed also in humans (FIG. 6B). Levels of interleukin 6 (IL-6) and TIMP Metallopeptidase Inhibitor 1 (TIMP-1) markers of inflammation and fibrosis measured in the supernatant before and after treatment with nonanal and 2-butanol (FIGS. 6C and 6D).

Microscopy image of cultured hepatocytes were stained with oil red O to show lipids. NASH hepatocytes as expected show a strong staining as shown in the Microscopy image in FIG. 7.

The data showed in FIGS. 6 and 7 indicate that hepatocytes culture was successful, and that healthy and NASH phenotype were generated.

Primary hepatocytes were treated with 2-butanol and nonanal diluted in the hepatocytes culture medium at a concentration of 50 ng/μL. Aliquots of the culture medium were collected at 0.5, 1, 2, 4, 6, and 24 hours. The remaining medium-compound mix that was not added to the cells was considered as 0 timepoint reference. Headspace analysis was performed by using Centri, an automated system used to collect VOCs in the headspace, coupled with a GC-MS Orbi-trap system, a high-resolution mass spectrometer for detection and quantification of VOCs. Peak identification of 2-butanol, 2-butanone, nonanal, and 1-nonanol was performed by comparing the peak against a reference standard. A p-value<0.05 was considered as statistically significant.

FIG. 8A. Levels of 2-butanol decrease over time in the absence of hepatocytes due to spontaneous evaporation. However, presence of NASH and healthy hepatocytes show a more marked reduction at 24 h. FIG. 8B. Levels of 2-butanone show no increase in the absence of hepatocytes. Healthy hepatocytes showed higher production of 2-butanone at 6 hours and the area under the peak for healthy is larger than for NASH, as expected given that the alcohol dehydrogenase pathway involved in the conversion of 2-butanol to 2-butanone is downregulated in NASH². FIG. 8C.

Nonanal showed a reduction in the absence of hepatocytes due to spontaneous evaporation. However, in the presence of hepatocytes, nonanal showed a more marked reduction. FIG. 8D. Nonanol was not produced in the absence of hepatocytes, while NASH hepatocytes showed higher production on nonanol compared to healthy hepatocytes (the area of the peak is larger in NASH than in healthy). This result aligns with the overexpression of AKR1B10, the enzyme that converts nonanal to nonanol, observed in NASH liver³.

We have shown for the first time that NASH-induced metabolic alterations are detectable using an exogenous volatile organic compound (EVOC) Probe, which is safe for human consumption and appears in breath after administration, together with its bioproduct. These data support that the use breath analysis using EVOC Probes for induced volatolomics for a NASH detection test which can be used to diagnose/detect NASH, monitoring progression or prognose NASH.

### REFERENCES

1. Endo, S. et al. Kinetic studies of AKR1B10, human aldose reductase-like protein: endogenous substrates and inhibition by steroids. Arch Biochem Biophys 487, 1-9, doi: 10.1016/j.abb.2009.05.009 (2009).
2. Li, H., Toth, E. & Cherrington, N. J. Alcohol Metabolism in the Progression of Human Nonalcoholic Steatohepatitis. Toxicol Sci 164, 428-438, doi: 10.1093/toxsci/kfyl06 (2018).
3. Kanno, M. et al. Serum aldo-keto reductase family 1 member B10 predicts advanced liver fibrosis and fatal complications of nonalcoholic steatohepatitis. J Gastroenterol 54, 549-557, doi: 10.1007/s00535-019-01551-3 (2019).

### Example 6. Detection of VOCs from NASH Rat Model

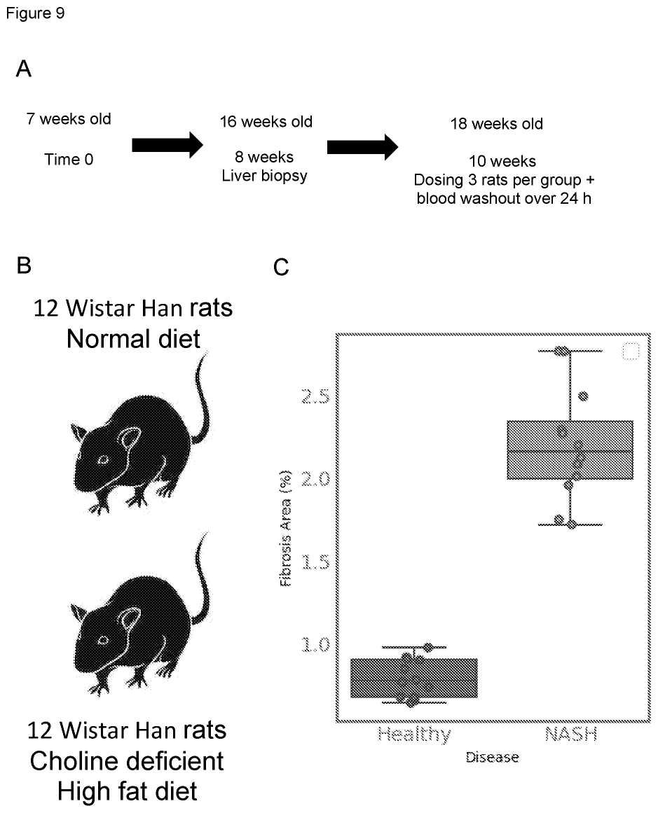

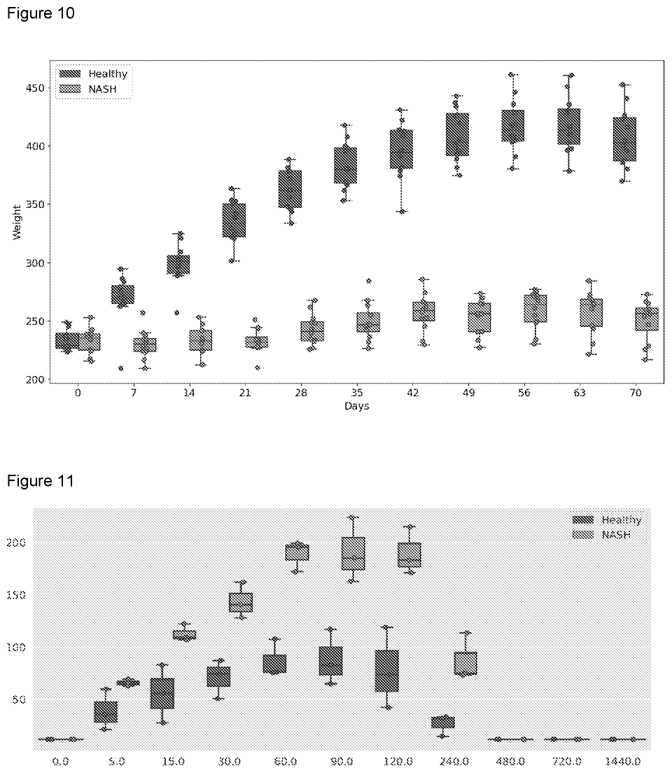

Several groups of three Wistar Han rats were fed a normal diet (ND) and similar number of groups of three rats a choline deficient high fat diet (CDHFD), which is a widely used treatment to induce liver fibrosis in rodent models¹. Fibrosis of the liver was assessed after 8 weeks and as expected CDHFD rats were found to have higher fibrosis compared to ND rats (FIG. 9A, B, and C). The rats on CDHFD showed less body weight increase compared to ND rats (FIG. 10). Ten weeks after the beginning of the diet rats were orally administered with either benzyl alcohol (208 mg/Kg) or 2-butanol (440 mg/Kg) or 2-pentanone (160 mg/Kg), and blood samples (200 μl) were collected before administration and after administration at the timepoints: 5, 15, 30 minutes, 1, 1.5, 2, 4, 8, 12, 24 hours.

Blood samples were analysed using gas chromatography mass spectrometry (GC/-MS) or HPLC to measure metabolitic bioproducts benzoic acid, 3-hydroxy-2-butanone, 2-3-butanediol, and 2,3-pentanediol.

Benzoic acid is a bioproduct of the alcohol dehydrogenase pathway, which is further metabolized to hippuric acid. Chronic liver damage induces a reduction of the hippuric acid metabolism². Consistent with these reports, we observed increased concentration of benzoic acid in the blood of CDHFD rats compared to ND rats after benzyl alcohol administration. In particular, benzoic acid was absent before administration. Levels increased in the blood from 5 to 240 minutes after administration with concentration found to be higher in rats on CDHFD. Blood concentrations reached undetectable levels in all the rats after 480 minutes (FIG. 11).

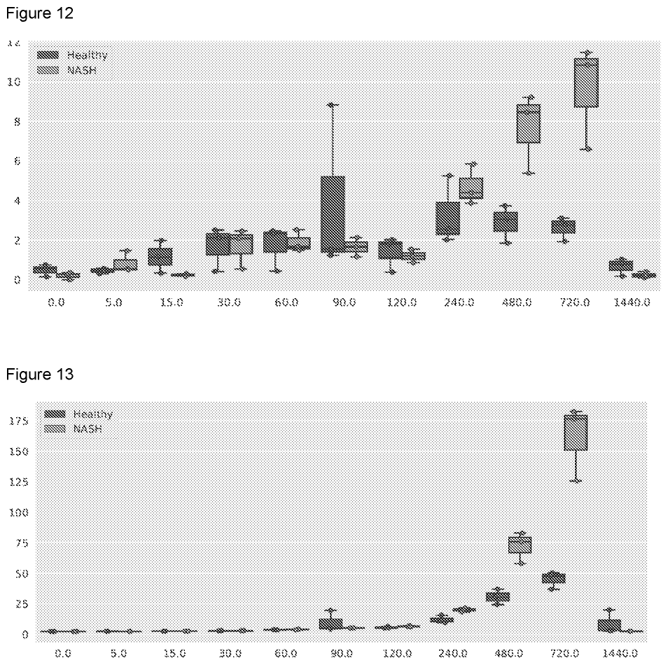

2-butanol is converted to 2-butanone by alcohol dehydrogenase. Then 2 butanone is converted to 3-hydroxy-2-butanone and 2,3-butanediol by CYP enzymes^{3,4}. We administered 2-butanol and found that concentration of 3-hydroxy-2-butanone and 2,3-butanediol increases 240 minutes after administration. At 8 and 12 hours (480, 720 minutes) after administration, these compounds showed higher levels in CDHFD compared to ND rats (FIGS. 12 and 13).

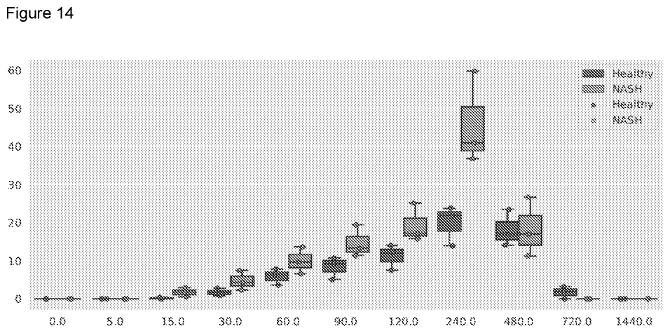

2-pentanone is converted to 2,3-pentanediol by CYPs⁴. After administration of 2-pentanone, blood levels of 2,3-pentanediol increased after 1 hour, with levels that became higher in CDHFD rats compared to ND rats after 4 hours (FIG. 14).

These data indicate that NASH induces hepatic alterations that change metabolism of tested compounds. These changes can be used for diagnostic purposes.

### Materials and Methods

All the bioanalytical experiments were conducted by WuXi AppTec (Nantong) Co., Ltd. Address: 699 South Huashi Road, Qidong, Natong, Jiangsu, P. R. China. Telephone: 0513-83395562. A total of 18 Wistar Han rats were acclimatized for 6 days. After that time, 9 rats remained on a normal diet, while 9 rats were switched to a choline deficient high fat diet (CDHFD) (L-Amino Acid Diet With 45 kcal % Fat With 0.1% Methionine, No Added Choline and 1% Cholesterol) (Research Diet, catalog #A16092003, https://researchdiets.com/en/formulas/a16092003). Rats had ad libitum access to food and water at all times unless differently specified. Body weight was measured every week.

After 8 weeks from the beginning of the diet, rats underwent a survivable liver biopsy procedure. Rats were deeply anesthetized with 3% isoflurane, a piece of liver from the left-lateral lobe was collected from a longitudinal incision (3-4 cm), immediately caudal of the xiphoid process and slightly to the animal's left side, in the abdominal skin. Animals were administered tolidine (2 mg/kg) and Penicillin (10 w U/Rat) subcutaneously at the end of the day of surgery and then once daily for 2-3 days as necessary and were monitored until full recovery. Collected liver tissue were sliced and stained with haematoxylin/eosin and fibrotic tissue was microscopically quantified as percentage of total area. Rats were allowed to recover from the biopsy for 2 weeks before further treatments.

Following 10 weeks of diet treatment, rats were randomised into groups each group composed of 3 healthy and 3 NASH rats for dosing with the different compounds.

Each group received an emulsion containing the compounds reported in table 1 at the indicated amount.

TABLE 1
Administered compounds and amount.

| | Amount | |
|---|---|---|
| | administered, | Group |
| Compound | mg/kg | number |
| 2-Pentanone | 160 | 1 |
| 2-Butanol | 440 | 2 |
| Benzyl alcohol | 208 | 3 |

Each compound preparation was administered as an emulsion containing 15% Tween80 diluted in water to deliver the target amount in 5 mL/kg body weight. The emulsion was administered by oral gavage after the rats were fasted for 16 hours.

Blood samples were collected pre (0 min) and post compound administration at the following timepoints: 5 min, 15 min, 30 min, 1 h, 1.5 h, 2 h, 4 h, 8 h, 12 h, and 24 h.

At each time point, ˜200 μL blood samples were collected into EDTA-K2 tubes and placed on wet ice until centrifugation. Plasma was generated, from the blood samples, by centrifugation at approximately 4° C., 3,200 g for 5 min.

A total of 100 μL of plasma was collected respectively and transferred into Eppendorf tubes. Of these, 95 μL of plasma were spiked with 400 μL of internal standard in acetonitrile, vortexed for 30s and centrifuged for 15 min at 3220 g. A total of 100 μL of supernatant was used for compounds quantification, unless differently specified.

Quantification of the compounds of interest was performed as indicated in Table 2.

TABLE 2
Compounds of interest quantification method.

| Compound | LLOQ (μg/mL) | Method |
|---|---|---|
| 3-hydroxy-2-butanone | 5 | GCMS |
| 2,3-pentanediol | 5 | GCMS |
| 2,3-butanediol | 5 | GCMS |
| Benzoic acid | 5 | HPLC |

Analytical method details are as follow:
3-hydroxy-2-butanone (Internal standard=Acetone):
2,3-butanediol and 2,3 pentanediol (Internal standard=1-heptanol)

TABLE 3
GC-MS analytical method/parameters for analysis of 3-hydroxy-
2-butanone, 2,3-pentanediol, and 2,3-butanediol

| Instrument | Agilent 8890GC System + 7000D GC/TQ | | |
|---|---|---|---|
| Column | DB-624 Ultra I 30 m × 0.25 mm, 1.4 μm | | |
| Oven | Rate(° C./min) | Rate(° C./min) | Rate(° C./min) |
| | — | 40 | 5 |
| | 20 | 160 | 5 |
| | 40 | 240 | 10 |

| Column flow | 0.8 | mL/min |
|---|---|---|
| Injection Mode | Split | |
| Split Ratio | 20:1 | |
| Inlet Temperature | 180° | C. |
| Injection Volume | 1 | μL |
| Aux Heaters | 250° | C. |
| Quad 1 Temp | 150° | C. |
| Quad 2 Temp | 150° | C. |
| Carrier Gas | He | |
| Run Time | 28 | min |
| Delay Time | 5 | min |
| Source | EI | |
| Source | 220° | C. |
| Temperature | | |

| | |
|---|---|
| Scan Mode | SIM |

Benzoic acid (Internal standard=hexanoic acid):
   Injected directly in the instrument.

TABLE 4
HPLC analytical method/parameters for analysis of benzoic acid

| | |
|---|---|
| Mobile Phase | A:0.1% TFA in H2O |
| | B:0.1% TFA in ACN |
| Acquisition Time(min) | 20 min |
| Injection Volume | 3 μL |
| Column | ACQUITY UPLC HSS T3 |
| | 2.1*100 mm, 1.8 um |
| Column Tem. (° C.) | 30 |
| Auto sample Tem.(° C.) | Room Temperature |

| Gradient | Time/min | B % |
|---|---|---|
| | 0 | 10 |
| | 10 | 90 |
| | 15 | 90 |
| | 15.1 | 10 |
| | 20 | 10 |

| | |
|---|---|
| Detector | DAD |
| Wavelength | 220 nm for benzoic acid |
| | 210 nm for benzyl alcohol |
| | 254 nm for benzaldehyde |

Response for each compound was normalized by the response of the internal standard, and absolute quantification was performed against a calibration curve.

### REFERENCES

1. Nevzorova Y A, Boyer-Diaz Z, Cubero F J, Gracia-Sancho J. Animal models for liver disease-A practical approach for translational research. J Hepatol 2020; 73 (2): 423-440. doi: 10.1016/j.jhep.2020.04.011. PMID: 32330604
2. Saltzman A, Caraway W T. Cinnamic acid as a test substance in the evaluation of liver function. J Clin Invest 1953; 32 (8): 711-719. doi: 10.1172/JCI102785. PMID: 13069619
3. Morey T E, Booth M, Wasdo S, Wishin J, Quinn B, Gonzalez D, et al. Oral adherence monitoring using a breath test to supplement highly active antiretroviral therapy. AIDS Behav 2013; 17 (1): 298-306. doi: 10.1007/s10461-012-0318-7. PMID: 23001413
4. Dietz F K, Rodriguez-Giaxola M, Traiger G J, Stella V J, Himmelstein K J. Pharmacokinetics of 2-butanol and its metabolites in the rat. J Pharmacokinet Biopharm 1981; 9 (5): 553-576. doi: 10.1007/BF01061026. PMID: 7334459

1. A method for detecting, staging, monitoring or prognosing non-alcoholic steatohepatitis (NASH) in a subject, comprising measuring the concentration of an exogenous substrate for an enzyme and/or measuring the concentration of a metabolite of said substrate in a biological sample obtained from said subject wherein the substrate is a generally recognised as safe (GRAS) compound and wherein the enzyme is an aldo-ketoreductase (AKR) or an alcohol dehydrogenase or a Cytochrome P450 (CYP) or aldehyde dehydrogenase or glycine N-acyltransferase.

2. The method according to claim 1, wherein the method further comprises determining the stage of NASH, wherein the NASH stage is selected from: NASH without fibrosis, NASH with fibrosis, NASH with hepatocellular carcinoma (HCC) or NASH with cirrhosis, such as decompensated cirrhosis.

3. The method according to claim 1, wherein the method is for detecting or prognosing NASH with fibrosis.

4. The method according to claim 1, wherein the enzyme is an aldo-ketoreductase (AKR).

5. The method according to claim 4 wherein the AKR is an AKR family 1 member.

6. The method according to claim 5 wherein said AKR is AKR1B10.

7. The method according to claim 1 wherein the substrate is selected from an aldehyde and/or an alcohol.

8. The method according to claim 1 wherein the substrate is nonanal and/or the metabolite is nonanol.

9. The method according to claim 8, wherein the substrate is 1-nonanal and/or the metabolite is 1-nonanol.

10. The method according to claim 1 wherein the substrate is trans-2-hexenal and/or the metabolite is trans-2-hexanol.

11. The method according to claim 1 wherein the substrate is hexanal and/or the metabolite is hexanol.

12. The method according to claim 1 wherein the substrate is benzaldehyde and/or the metabolite is benzyl alcohol.

13. The method according to claim 1 wherein the substrate is citral and/or the metabolite is nerol.

14. The method according to claim 1, wherein the enzyme is an alcohol dehydrogenase.

15. The method according to claim 14, wherein the substrate is butanol and/or the metabolite is butanone.

16. The method according to claim 15, wherein the substrate is 2-butanol and/or the metabolite is 2-butanone.

17. The method according to claim 14, wherein the substrate is 2-pentanone.

18. The method according to claim 14, wherein the substrate is 2-pentanone and/or the metabolite is 2-pentanol, 3-hydroxy-2-pentanone or 2,3-pentanediol.

19. The method according to claim 14, wherein the substrate is benzyl alcohol and the metabolites are benzylaldehyde and/or benzoic acid.

20. The method according to claim 14, wherein the enzyme is aldehyde dehydrogenase

21. The method according to claim 20, wherein the substrate is benzaldehyde and the metabolite is benzoic acid.

22. The method according to claim 1, wherein the enzyme is a Cytochrome P450 (CYP).

23. The method according to claim 22, wherein the CYP is CYP1A1, CYP1A2, CYP1 B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7 or CYP3A43. In one embodiment, the enzyme is CYP2C19, CYP2C9 and/or CYP3A4

24. The method according to claim 22, wherein the substrate is 2-butanone and the metabolite is 3-hydroxy-2-butanone and/or 2,3-butanediol.

25. The method according to claim 22, wherein the substrate is 2-pentanone and the metabolite is 2,3-pentanediol.

26. The method according to claim 1, wherein the enzyme is glycine N-acyltransferase.

27. The method according to claim 26, wherein the substrate is benzoic acid and the metabolite is hippuric acid.

28. The method according to claim 1 wherein the substrate is labelled.

29. The method according to claim 28 wherein said label is 12C, 13C, 14C, 2H, 14N or 180.

30. The method according to claim 1 wherein the substrate is not labelled.

31. The method according to claim 1 wherein the biological sample is selected from breath, urine, blood, serum, and/or tissue.

32. The method according to claim 1 wherein said method comprises establishing a test subject value based on a concentration of said substrate or metabolite in said test subject or a combination thereof.

33. The method according to claim 32 wherein said test subject value is compared to one or more reference values and wherein a difference in the test subject value and a reference value indicates a likelihood of NASH.

34. The method according to claim 33 wherein said reference value is the value of a subject that has been diagnosed with NASH.

35. The method according to claim 33 wherein said reference value is the value of a subject that has been diagnosed with non-alcoholic fatty liver disease (NAFLD).

36. The method according to claim 33 wherein said reference value is the value of a subject with NASH that will progress to decompensated cirrhosis or HCC.

37. The method according to claim 33 wherein said reference value is the value of a healthy subject.

38. The method according to claim 1 wherein the concentration of two or more exogenous substrates for the enzyme and/or the concentration of two or more metabolites is measured.

39. The method according to claim 1, wherein the subject has been administered the exogenous substrate for the enzyme.

40. The method according to claim 1 wherein the concentration of the metabolite is measured.

41. A method for determining efficacy of a treatment comprising in a subject diagnosed with NASH, assessing the activity of an enzyme by measuring the concentration of an exogenous substrate for said enzyme and/or measuring the concentration of a metabolite of said substrate in a biological sample obtained from the subject, wherein said subject has received treatment for NASH and wherein the enzyme is an aldo-ketoreductase (AKR) or an alcohol dehydrogenase or a Cytochrome P450 (CYP).

42. The method according to claim 41, wherein the method comprises analysing a first biological sample obtained from said subject at a first time point, and then analysing one or more additional biological samples obtained from said subject at one or more additional time points or ratios thereof.

43. The method according to claim 41, wherein said treatment of NASH is gastric bypass surgery, and/or a drug-based treatment comprising the administration of at least one drug selected from statins, incretin analogues, metformin, rimonabant, thiazolidinediones, and orlistat.

44. A method of monitoring the progression of NASH in a subject, comprising measuring the concentration of an exogenous substrate for an enzyme and/or measuring the concentration of a metabolite of said substrate in a biological sample obtained from a subject wherein the substrate is a generally recognised as safe (GRAS) compound and wherein the enzyme is an aldo-ketoreductase (AKR) or an alcohol dehydrogenase or a Cytochrome P450 (CYP).

45. A kit for the detection or prognosis of NASH comprising substrate for an enzyme and/or the metabolite of said substrate and a device for capturing a biological sample from a patient.

46. The kit of claim 45 wherein said substrate and/or metabolite are selected from nonanal, buntanol, trans-2-hexenal, hexanal, benzaldehyde, citral nonanone, butanone trans-2-hexenol, hexenol, benzyl alcohol, nerol, 3-hydroxy-2-butanone, 2,3-butanediol, benzoic acid, hippuric acid, 2-pentanone and 2,3-pentanediol.

47. A use of an exogenous substrate and or metabolite for an enzyme whose activity or expression is upregulated or downregulated in NASH in a method for detecting or prognosing NASH, wherein said substrate is selected from nonanal, butanol, trans-2-hexenal, hexanal, benzaldehyde, citral, benzoic acid, butanone, and 2-pentanone, and said metabolite is selected from nonanol, butanone, trans-2-hexenol, hexanol, benzyl alcohol, nerol, 2-pentanone, 3-hydroxy-2-butanone, 2,3-butanediol, hippuric acid, and 2,3-pentanediol.

48. Nonanal, butanol, trans-2-hexenal, hexanal, benzaldehyde, citral, 2-pentanone, nonanone, butanone trans-2-hexenol, hexanol, benzyl alcohol, nerol, 2-pentanol, benzoic acid, hippuric acid, 2,3-butanediol, 3-hydroxy-2-pentanone and/or 2,3-pentanediol for use in an in vivo method of detecting or prognosing NASH in a subject, comprising measuring the concentration of nonanal, buntanol, trans-2-hexenal, hexanal, benzaldehyde, citral, 2-pentanone, nonanol, butanone, trans-2-hexenol, hexanol, benzyl alcohol, nerol 2-pentanol, benzoic acid, hippuric acid, 2,3-butanediol, 3-hydroxy-2-pentanone and/or 2,3-pentanediol in a biological sample obtained from said subject.

49. The use of nonanal, buntanol, trans-2-hexenal, hexanal, benzaldehyde, citral, 2-pentanone nonanol, butanone trans-2-hexenol, hexanol, benzyl alcohol, nerol, 2-pentanol, benzoic acid, hippuric acid, 2,3-butanediol, 3-hydroxy-2-pentanone and/or 2,3-pentanediol as a biomarker for NASH.

50. A method of differentiating between NASH and other stages of NAFLD in a subject, comprising measuring the concentration of an exogenous substrate for an enzyme and/or measuring the concentration of a metabolite of said substrate in a biological sample obtained from a subject wherein the substrate is a generally recognised as safe (GRAS) compound.

51. The method according to claim 50, wherein activity or expression of said enzyme is upregulated or downregulated in NASH.

52. The method according to claim 50, wherein the enzyme is an aldo-ketoreductase (AKR) or an alcohol dehydrogenase or a Cytochrome P450 (CYP).

53. A method for detecting or prognosing early stage non-alcoholic steatohepatitis (NASH) in a subject, comprising measuring the concentration of an exogenous substrate for an enzyme and/or measuring the concentration of a metabolite of said substrate in a biological sample obtained from said subject wherein the substrate is a generally recognised as safe (GRAS) compound.

54. The method according to claim 53, wherein the enzyme is an aldo-ketoreductase (AKR) or an alcohol dehydrogenase or a Cytochrome P450 (CYP).

55. A method of determining the stage of NASH in a subject, comprising measuring the concentration of an exogenous substrate for an enzyme and/or measuring the concentration of a metabolite of said substrate in a biological sample obtained from a subject wherein the substrate is a generally recognised as safe (GRAS) compound.

56. The method of claim 55 wherein the activity or expression of said enzyme is upregulated or downregulated in NASH.

57. The method of claim 56 wherein the enzyme is an aldo-ketoreductase (AKR) or an alcohol dehydrogenase or a Cytochrome P450 (CYP).

58. A method for detecting or prognosing NASH in a subject, detecting or prognosing early stage NASH in a subject, determining the stage of NASH in a subject or differentiating between NASH and other stages of NAFLD in a subject comprising measuring the concentration of an exogenous substrate for an enzyme and/or measuring the concentration of a metabolite of said substrate in a biological sample obtained from said subject wherein the substrate is a generally recognised as safe (GRAS) compound and wherein the enzyme is not a CYP enzyme.

59. A method for detecting or prognosing NASH in a subject, detecting or prognosing early stage NASH in a subject, determining the stage of NASH in a subject or differentiating between NASH and other stages of NAFLD in a subject comprising measuring the concentration of an exogenous substrate for an enzyme and/or measuring the concentration of a metabolite of said substrate in a biological sample obtained from said subject wherein the substrate is a generally recognised as safe (GRAS) compound wherein the substrate is not limonene.

60. A method for determining efficacy of a treatment comprising in a subject diagnosed with NASH, assessing the activity of an enzyme by measuring the concentration of an exogenous substrate for said enzyme and/or measuring the concentration of a metabolite of said substrate in a biological sample obtained from the subject, wherein the enzyme is not a CYP enzyme or wherein the substrate is not limonene.
